(19) United States
(12) Patent Application Publication
(10) Pub. No.: US 2014/0196172 A1
(43) Pub. Date: Jul. 10, 2014

(54) Organelle Targeting Nanocarriers

(71) Applicant: Her Majesty the Queen in Right of Canada, as Repre sented by the Minister of Agriculture and AgriFoo, Lethbridge (CA)

(72) Inventors: Francois Eudes, Lethbridge (CA); Trevor Macmillan, Lethbridge (CA)

(21) Appl. No.: 14/129,386

(22) PCT Filed: Aug. 2, 2012
(86) PCT No.: PCT/CA2012/000727

§ 371 Date: Mar. 24, 2014

Related U.S. Application Data
(60) Provisional application No. 61/514,988, filed on Aug. 4, 2011.

(51) Int. Cl. C12N 15/82

(52) U.S. Cl. CPC C12N 15/8201
USPC 800/278; 435/455; 435/468; 435/419; 530/324; 530/326; 530/327; 800/298

(57) ABSTRACT

Provided are organelle targeting nanocarriers, including peptides, which act to deliver biological molecules such as nucleic acids to non-nuclear organelles such as mitochondria and chloroplasts. Also provided are methods for genetic transformation of non-nuclear organelles using such nanocarriers.

### FIELD OF THE INVENTION

The present application is directed to compositions and methods for delivering biological molecules, such as proteins and nucleic acids, to non-nuclear organelles. More specifically, the present application describes compositions and methods for genetic transformation of mitochondria and chloroplasts.

### BACKGROUND OF THE INVENTION

Alternative agricultural molecular biotechnology techniques are needed to genetically engineer globally important food and industrial crops to meet the world demand for these renewable resources. Unfortunately, plants are recalcitrant to most of the genetic transformation methods developed to manipulate mammalian cells. However, peptide transfection technology is currently emerging as a viable plant transfection technology.

Cell penetrating peptides (CPPs) are short cationic peptides which are capable of transducing polar hydrophilic compounds such as nucleic acids across cell membranes in a receptor independent manner (Veldhoen, S., Recent Developments in Peptide-Based Nucleic Acid Delivery. International Journal of Molecular Sciences (2008) 9(7): 1276-1320). An example of such a cell penetrating peptide is HIV-1 Tat 49-57 (RKKRRQRRR) (Vives, E., P. Brodin, and B. Lebleu, A Truncated HIV-1 Tat Protein Basic Domain Rapidly Translocates through the Plasma Membrane and Accumulates in the Cell Nucleus. J. Biol. Chem. (1997) 272(25): 16010-16017; Wender, P. A., et al., The design, synthesis, and evaluation of molecules that enable or enhance cellular uptake: Peptoid molecular transporters. Proceedings of the National Academy of Sciences (2000) 97(24): 13003-13008).

The Tat sequence includes basic amino acids that allow Tat to transduce itself and attached cargo across the outer plasma membrane of cells. The Tat-cargo complex accumulates in the nucleus of cells due to the presence of a subcellular localization sequence within its peptide sequence called a nuclear localization signal (NLS) (Nagahara, H., et al., Transduction of full-length TAT fusion proteins into mammalian cells: TAT-p27Kip1 induces cell migration. Nat Med (1998) 4(12): 1449-1452).

Such subcellular localization sequences found on the N-terminus of proteins are referred to collectively as protein sorting signal sequences. Each protein sorting signal sequence is a distinct peptide sequence that targets nascent proteins translated in the cytosol to a specific subcellular location within cells. Protein sorting signals include nuclear localization signals (NLS), which target the nucleus, mitochondrial targeting peptides (mTP), which target mitochondria, and chloroplast transit peptides (cTP), which target chloroplasts. cTPs, mTPs and NLS are recognized by translocation machinery that facilitates the transport of cytosolic proteins containing these sequences across the double membrane into specific organelles (Emanuelsson, O., et al., Locating proteins in the cell using TargetP, SignalP and related tools. Nat. Protocols (2007) 2(4): 953-971).

Specific structural and chemical properties that facilitate efficient cellular uptake and mitochondrial accumulation have been applied to the development of mitochondrial therapeutic agents and cancer drugs. For example, synthetic peptides that incorporate delocalized lipophilic cations (DLCs) have been prepared (Fernandez-Carneado, J., et al., Highly Efficient, Nonpeptidic Oligoguanidinium Vectors that Selectively Internalize into Mitochondria, Journal of the American Chemical Society (2004) 127(3): 869-874). In addition, cell-permeable antioxidant peptides used to reduce cellular oxidative stress caused by reactive oxygen species in the inner mitochondrial membrane have been developed (Zhao, K., et al., Cell-permeable Peptide Antioxidants Targeted to Inner Mitochondrial Membrane inhibit Mitochondrial Swelling, Oxidative Cell Death, and Reperfusion Injury, J. Biol. Chem. (2004) 279(33): 34682-34690). These peptides possess a structural motif including alternating synthetic aromatic residues conferring antioxidant properties and basic amino acids conferring cell permeant properties. Recently, mitochondrial penetrating peptides (MPPs) based on this previously identified structural motif of alternating aromatic and basic residues have been developed, and the properties of DLCs have been incorporated into specific sites within these peptides (Horton, K. L. et al, Mitochondria-Penetrating Peptides, Chemistry & Biology (2008) 15: 375-382).

Certain organelles, such as mitochondria and chloroplasts, contain DNA which is expressed, but which is generally inherited from only one parent, unlike the nuclear genome. Mitochondrial genes are generally inherited maternally and in most flowering plants, for example, chloroplasts are not inherited from the male parent. Because of this, such organelles have been a target for genetic transformation, especially in plants, since any transformed genes are more likely to be biologically contained and not disseminated by pollen, therefore posing a lower environmental risk. Furthermore, dysfunction of mitochondria has been associated with specific diseases for which genetic and other therapies delivered directly to mitochondria might be useful treatments.

Therefore, new methods for genetically transforming organisms by selectively introducing genetic material into the genome of organelles such as mitochondria and chloroplasts are desirable.

### SUMMARY OF THE INVENTION

One aspect of the present invention provides a method for delivering a nucleic acid to a non-nuclear organelle in a cell, the method comprising exposing the cell to a composition comprising at least one nucleic acid and at least one organelle targeting nanocarrier; wherein the at least one nucleic acid translocates across the cell membrane and enters the non-nuclear organelle in the presence of the at least one organelle targeting nanocarrier. In at least one embodiment, the cell is a plant cell. In at least one embodiment, the plant cell is selected from an embryogenic somatic cell, a protoplast and a microspore. In at least one embodiment, the cell is an animal cell. In at least one embodiment, the nucleic acid is DNA.

In at least one embodiment, the non-nuclear organelle is a mitochondrion. According to such embodiments, the organelle targeting nanocarrier can be a polypeptide having a charge ratio of about 4 to about 7 and hydrophilicity of about 0 to about −0.5. Alternatively in such embodiments, the organelle targeting nanocarrier can be a polypeptide having a sequence selected from:

| | |
|---|---|
| MFSYLPRYPLRAASARALVRATRPSYRSALLRYQ; | (SEQ ID NO: 1) |
| MAAWMRSLFSPLKKLWIRMH; | (SEQ ID NO: 2) |
| MKLLWRLILSRKW; | (SEQ ID NO: 3) |
| MWWRRSRTNSLRYT; | (SEQ ID NO: 4) |
| and | |
| MLFRLRRSVRLRGLLA. | (SEQ ID NO: 5) |

In at least one embodiment, the non-nuclear organelle is a chloroplast. According to such embodiments, the organelle targeting nanocarrier can be a polypeptide having a charge ratio of about 2 to about 4.2 and hydrophilicity of about 0 to about −0.2. Alternatively in such embodiments, the organelle targeting nanocarrier can be a polypeptide having a sequence selected from:

| | |
|---|---|
| MGGCVSTPKSCVGAKLR; | (SEQ ID NO: 6) |
| MQTLTASSSVSSIQRHRPHPAGRRSSSVTFS; | (SEQ ID NO: 7) |
| MKNPPSSFASGFGIR; | (SEQ ID NO: 8) |
| MAALIPAIASLPRAQVEKPHPMPVSTRPGLVS; | (SEQ ID NO: 9) |
| and | |
| MSSPPPLFTSCLPASSPSIRRDSTSGSVTSPLR. | (SEQ ID NO: 10) |

In another aspect of the present invention, there is provided a method for producing a genetically modified plant cell, the method comprising exposing a plant cell containing a non-nuclear organelle to a composition comprising at least one nucleic acid and at least one organelle targeting nanocarrier; wherein the at least one nucleic acid translocates across a cell membrane of the cell and enters the non-nuclear organelle in the presence of the at least one organelle targeting nanocarrier so as to transfect the non-nuclear organelle. In at least one embodiment, the plant cell is an embryogenic microspore.

Another aspect of the present invention provides a genetically modified plant cell produced by the method described herein.

In another aspect of the present invention, there is provided a method for producing a genetically modified plant, the method comprising exposing a plant cell containing a non-nuclear organelle to a composition comprising at least one nucleic acid and at least one organelle targeting nanocarrier: wherein the at least one nucleic acid translocates across a cell membrane of the cell and enters the non-nuclear organelle in the presence of the at least one organelle targeting nanocarrier so as to transfect the non-nuclear organelle; and generating a plant from the plant cell containing the transfected non-nuclear organelle. In at least one embodiment, the plant cell is an embryogenic microspore.

A further aspect of the present invention provides a genetically modified plant produced by the method described herein. Yet another aspect provides a seed of such a genetically modified plant, the seed containing a transfected non-nuclear organelle as described herein.

In another aspect of the present invention, there is provided a method for producing a genetically modified animal cell, the method comprising exposing an animal cell containing at least one mitochondrion to a composition comprising at least one nucleic acid and at least one mitochondrion targeting nanocarrier; wherein the at least one nucleic acid translocates across a cell membrane of the cell and enters the at least one mitochondrion in the presence of the at least one mitochondrion targeting nanocarrier so as to transfect the at least one mitochondrion. In at least one embodiment, the animal cell is a mammalian cell. In at least one embodiment, the animal cell is a human cell.

Another aspect of the present invention provides a genetically modified animal cell produced by the method described herein.

An additional aspect of the present invention provides a mitochondrion targeting nanocarrier wherein the mitochondrion targeting nanocarrier is a polypeptide having a mitochondrial targeting peptide (mTP) sequence, a charge ratio of about 4 to about 7 and a hydrophilicity of about 0 to about −0.5. In at least one embodiment, the polypeptide has a sequence selected from:

| | |
|---|---|
| MFSYLPRYPLRAASARALVRATRPSYRSALLRYQ; | (SEQ ID NO: 1) |
| MAAWMRSLFSPLKKLWIRMH; | (SEQ ID NO: 2) |
| MKLLWRLILSRKW; | (SEQ ID NO: 3) |
| MWWRRSRTNSLRYT; | (SEQ ID NO: 4) |
| and | |
| MLFRLRRSVRLRGLLA. | (SEQ ID NO: 5) |

Another aspect of the present invention provides a chloroplast targeting nanocarrier wherein the chloroplast targeting nanocarrier is a polypeptide having a chloroplast transit peptide (cTP) sequence, a charge ratio of about 2 to about 4.2 and a hydrophilicity of about 0 to about −0.2. In at least one embodiment, the polypeptide has a sequence selected from:

| | |
|---|---|
| MGGCVSTPKSCVGAKLR; | (SEQ ID NO: 6) |
| MQTLTASSSVSSIQRHRPHPAGRRSSSVTFS; | (SEQ ID NO: 7) |
| MKNPPSSFASGFGIR; | (SEQ ID NO: 8) |
| MAALIPAIASLPRAQVEKPHPMPVSTRPGLVS; | (SEQ ID NO: 9) |
| and | |
| MSSPPPLFTSCLPASSPSIRRDSTSGSVTSPLR. | (SEQ ID NO: 10) |

### BRIEF DESCRIPTION OF THE DRAWINGS

These and other features of the present invention will become apparent from the following description and claims, and the accompanying drawings in which:

FIG. 1A is a confocal microscopic image (Nikon) of a cross section of a triticale protoplast showing fluorescence from fluorescein-labeled cTP1 (SEQ ID NO: 6);

FIG. 1B is a confocal microscopic image (Nikon) of a cross section of a triticale protoplast showing both fluorescence from fluorescein-labeled cTP1 (SEQ ID NO: 6) and chloroplast autofluorescence;

FIG. 2A is a confocal microscopic image (Nikon) of a triticale protoplast showing fluorescence from fluorescein-labeled mTP3 (SEQ ID NO: 3);

FIG. 2B is a confocal microscopic image (Nikon) of a triticale protoplast showing fluorescence from MitoTracker® Orange;

FIG. 2C is a confocal microscopic image (Nikon) of a triticale protoplast showing both fluorescence from fluorescein-labeled mTP3 (SEQ ID NO: 3) and fluorescence from MitoTracker® Orange;

FIG. 3A is a confocal microscopic image (Olympus) of a cross section of a tobacco protoplast showing chloroplast autofluorescence;

FIG. 3B is a confocal microscopic image (Olympus) of a cross section of a tobacco protoplast showing fluorescence from fluorescein-labeled mTP3 (SEQ ID NO: 3);

FIG. 3C is a confocal microscopic image (Olympus) of a cross section of a tobacco protoplast showing fluorescence from MitoTracker® Orange;

FIG. 3D is a confocal microscopic image (Olympus) of a cross section of a tobacco protoplast showing chloroplast autofluorescence, fluorescence from fluorescein-labeled mTP3 (SEQ ID NO: 3) and fluorescence from MitoTracker® Orange;

FIG. 4A is a stacked depth confocal microscopic image (Olympus) of a tobacco protoplast showing chloroplast autofluorescence;

FIG. 4B is a stacked depth confocal microscopic image (Olympus) of a tobacco protoplast showing fluorescence from fluorescein-labeled cTP1 (SEQ ID NO: 6);

FIG. 4C is a stacked depth confocal microscopic image (Olympus) of a tobacco protoplast showing fluorescence from MitoTracker® Orange;

FIG. 4D is a stacked depth confocal microscopic image (Olympus) of a tobacco protoplast showing chloroplast autofluorescence, fluorescence from fluorescein-labeled cTP1 (SEQ ID NO: 6) and fluorescence from MitoTracker® Orange;

FIG. 5A is a confocal microscopic image (Nikon) of a microspore showing fluorescence from fluorescein-labeled mTP3 (SEQ ID NO: 3);

FIG. 5B is a confocal microscopic image (Nikon) of a microspore showing both fluorescence from fluorescein-labeled mTP3 (SEQ ID NO: 3) and fluorescence from MitoTracker® Orange;

FIG. 6A is a confocal microscopic image (Nikon) of a microspore showing fluorescence from fluorescein-labeled mTP1 (SEQ ID NO: 1);

FIG. 6B is a confocal microscopic image (Nikon) of a microspore showing fluorescence from MitoTracker® Orange;

FIG. 6C is a confocal microscopic image (Nikon) of a microspore showing both fluorescence from fluorescein-labeled mTP1 (SEQ ID NO: 1) and fluorescence from MitoTracker® Orange;

FIG. 7 is a confocal microscopic image (Nikon) of MDCK (Madin-Darby canine kidney) cells showing fluorescence from fluorescein-labeled mTP1 (SEQ ID NO: 1);

FIG. 8A is a confocal microscopic image (Nikon) of MDCK cells showing fluorescence from fluorescein-labeled mTP1 (SEQ ID NO: 1);

FIG. 8B is a confocal microscopic image (Nikon) of MDCK cells showing fluorescence from MitoTracker® Orange;

FIG. 8C is a confocal microscopic image (Nikon) of MDCK cells showing both fluorescence from fluorescein-labeled mTP1 (SEQ ID NO: 1) and fluorescence from MitoTracker® Orange;

FIG. 9A is a confocal microscopic image (Nikon) of MDCK cells showing fluorescence from fluorescein-labeled mTP5 (SEQ ID NO: 5);

FIG. 9B is a confocal microscopic image (Nikon) of MDCK cells showing fluorescence from MitoTracker® Orange;

FIG. 9C is a confocal microscopic image (Nikon) of MDCK cells showing both fluorescence from fluorescein-labeled mTP5 (SEQ ID NO: 5) and fluorescence from MitoTracker® Orange;

FIG. 10 is a confocal microscopic image (Nikon) of MDCK cells showing fluorescence from fluorescein-labeled mTP4 (SEQ ID NO: 4);

FIG. 11 is a map of the reporter plasmid pWMaadAGFP;

FIG. 12 is a map of the reporter plasmid pWCaadAGFP;

FIG. 13A is a confocal microscopic image (Nikon) of a triticale protoplast transfected with the pWMaadA16GFP plasmid in the presence of mTP4 (SEQ ID NO: 4), showing fluorescence from green fluorescent protein (GFP);

FIG. 13B is a confocal microscopic image (Nikon) of a triticale protoplast transfected with the pWMaadA16GFP plasmid in the presence of mTP4 (SEQ ID NO: 4), showing fluorescence from both GFP and MitoTracker® Orange;

FIG. 13C is a confocal microscopic image (Nikon) of a triticale protoplast transfected with the pWMaadA16GFP plasmid in the presence of mTP4 (SEQ ID NO: 4), showing fluorescence from both GFP and MitoTracker® Orange, and chloroplast autofluorescence;

FIG. 14A is a confocal microscopic image (Nikon) of a triticale protoplast transfected with the pWMaadA16GFP plasmid in the presence of mTP2 (SEQ ID NO: 2), showing fluorescence from GFP;

FIG. 14B is a confocal microscopic image (Nikon) of a triticale protoplast transfected with the pWMaadA16GFP plasmid in the presence of mTP2 (SEQ ID NO: 2), showing fluorescence from MitoTracker® Orange;

FIG. 14C is a confocal microscopic image (Nikon) of a triticale protoplast transfected with the pWMaadA16GFP plasmid in the presence of mTP2 (SEQ ID NO: 2), showing fluorescence from both GFP and MitoTracker® Orange;

FIG. 15 is a confocal microscopic image (Nikon) of Caco-2 cells transfected with the pWMaadA16GFP plasmid in the presence of mTP1 (SEQ ID NO: 1), showing fluorescence from both GFP and MitoTracker® Orange;

FIG. 16 is a confocal microscopic image (Nikon) of F1112 cells transfected with the pWMaadA16GFP plasmid in the presence of mTP1 (SEQ ID NO: 1), showing fluorescence from both GFP and MitoTracker® Orange;

FIG. 17 is a plot showing the level of expression (fold increase, average of 4 replicates) of GFP in triticale microspores transfected with the pWMaadA16GFP plasmid in the presence of mTP1 (SEQ ID NO: 1), mTP2 (SEQ ID NO: 2), mTP3 (SEQ ID NO: 3), mTP4 (SEQ ID NO: 4) or mTP5 (SEQ ID NO: 5) compared to the level of expression (fold increase) of the internal control elongation factor 1a (EF1a), as measured by quantitative real time PCR of mRNA levels;

FIG. 18 is a plot showing the level of expression (fold increase, average of 4 replicates) of GFP in triticale microspores transfected with the pWCaadA16GFP plasmid in the presence of cTP1 (SEQ ID NO: 6), cTP2 (SEQ ID NO: 7), cTP3 (SEQ ID NO: 8), cTP4 (SEQ ID NO: 9) or cTP5 (SEQ ID NO: 10) compared to the level of expression (fold increase) of the internal control elongation factor 1a (EF1a), as measured by quantitative real time PCR of mRNA levels;

FIG. 19 is a plot showing the level of expression (fold increase, average of 4 replicates) of GFP in triticale protoplasts transfected with the pWMaadA16GFP plasmid in the presence of mTP1 (SEQ ID NO: 1), mTP2 (SEQ ID NO: 2), mTP3 (SEQ ID NO: 3), mTP4 (SEQ ID NO: 4) or mTP5 (SEQ ID NO: 5) compared to the level of expression (fold increase) of the internal control elongation factor 1a (EF1a), as measured by quantitative real time PCR of mRNA levels; and

FIG. 20 is a plot showing the level of expression (fold increase, average of 4 replicates) of GFP in triticale protoplasts transfected with the pWCaadA16GFP plasmid in the presence of cTP1 (SEQ ID NO: 6), cTP2 (SEQ ID NO: 7), cTP3 (SEQ ID NO: 8), cTP4 (SEQ ID NO: 9) or cTP5 (SEQ ID NO: 10) compared to the level of expression (fold increase) of the internal control elongation factor 1a (EF1a), as measured by quantitative real time PCR of mRNA levels.

### DETAILED DESCRIPTION OF THE INVENTION

One aspect of the present invention provides a method for delivering a nucleic acid to a non-nuclear organelle in a cell. In at least one embodiment, the cell is a plant cell, including but not limited to somatic cells, embryogenic somatic cells, mesophyll protoplasts and microspores. In at least one embodiment, the cell is an animal cell, including but not limited to a mammalian cell. In at least one embodiment, the cell is a human cell.

The nucleic acid is delivered to a subcellular non-nuclear organelle in the cell. Desirable target non-nuclear organelles are those which contain endogenous nucleic acids, including but not limited to genomic DNA, and which can express one or more genes from the endogenous nucleic acids. In at least one embodiment the organelle is a chloroplast. In at least one embodiment, the organelle is a mitochondrion.

The cell is exposed to a composition comprising at least one nucleic acid and at least one organelle targeting nanocarrier. Desirably, the nucleic acid can be expressed in the non-nuclear organelle or can transform the genome of the non-nuclear organelle. The nucleic acid can be RNA or DNA, and can be a naturally occurring or an artificial nucleic acid. As used herein, the term “artificial nucleic acid” is intended to mean a nucleic acid (RNA or DNA) which has been artificially or synthetically produced or modified. In at least one embodiment, the nucleic acid comprises DNA. In at least one embodiment, the nucleic acid comprises one or more genes expressible in the target non-nuclear organelle. In at least one embodiment, the nucleic acid comprises a plasmid, artificial chromosome, or gene construct. The person of skill in the art will be aware of suitable nucleic acids, and of methods for selecting and preparing such nucleic acids.

In at least one embodiment, the nucleic acid further comprises a marker gene. As used herein, the term “marker gene” is intended to mean a gene encoding a gene product whose presence can be detected and/or measured when expressed. Marker genes are well known in the art and include but are not limited to genes encoding proteins whose presence can be detected and measured by chemical or biochemical means, and genes encoding proteins that are detectable and/or measurable by their physical properties. Genes encoding proteins whose presence can be detected and measured by chemical or biochemical means include but are not limited to genes encoding enzymes and the like, and genes whose expression is associated with antibiotic resistance. Genes encoding proteins that are detectable and/or measurable by their physical properties include but are not limited to genes encoding proteins which are detectable by fluorescence, such as Aequorea victoria green fluorescent protein (GFP) and the like. It will be understood by one skilled in the art that a marker gene can be used to select for cells stably expressing the marker gene. For example, when the marker gene is a gene associated with resistance to an antibiotic, cells expressing the marker gene can be selected for by growing the cells in the presence of an amount of the antibiotic that would be lethal to the cells in the absence of expression of the marker gene.

In at least one embodiment, the organelle targeting nanocarrier is a polypeptide which can target one or more subcellular non-nuclear organelles. In at least one embodiment, the nanocarrier polypeptide includes an N-terminal protein sorting signal sequence. In at least one embodiment, the nanocarrier polypeptide includes an N-terminal protein sorting signal sequence which is specific for a subcellular non-nuclear organelle as described herein. In at least one embodiment, the N-terminal protein sorting signal sequence is a chloroplast transit peptide (cTP) sequence. In at least one embodiment, the N-terminal protein sorting signal sequence is a mitochondrial targeting peptide (mTP) sequence. In at least one embodiment, the N-terminal protein sorting signal sequence is a sequence found naturally in at least one protein of at least one plant.

Without wishing to be bound by theory, it is presently believed that the organelle targeting nanocarrier interacts with the membrane of the non-nuclear organelle so as to facilitate the entry of the nucleic acid inside the non-nuclear organelle. The organelle targeting nanocarrier may or may not itself enter the non-nuclear organelle, and it will be understood by the person of skill in the art that such entry of the organelle targeting nanocarrier itself into the non-nuclear organelle is not a requirement in order for the nucleic acid to enter the non-nuclear organelle in the presence of the organelle targeting nanocarrier.

In at least one embodiment, the organelle targeting nanocarrier polypeptide also has cell penetrating properties. In at least one embodiment, the polypeptide comprises no more than 100 amino acid residues. In at least one embodiment, the polypeptide comprises no more than 35 amino acid residues. In at least one embodiment, the polypeptide comprises from about 5 to about 35 amino acid residues.

As used herein, the term “net cationic charge” in reference to a polypeptide is defined as the net charge Z of a peptide calculated at pH 7.0, using the following formula:

$$Z = {\sum }_{i} {N}_{i} \frac{{10}^{p K {a}_{i}}}{{10}^{pH} + {10}^{p K {a}_{i}}} - {\sum }_{j} {N}_{j} \frac{{10}^{pH}}{{10}^{pH} + {10}^{p K {a}_{j}}}$$

wherein Nᵢ is the number of the ith basic group in the peptide (the N-terminal amino group and side chains of arginine, lysine and histidine residues); pKaᵢ is the pKₐ value of the ith basic group; Nⱼ is the number of the jth acidic group in the peptide (the C-terminal carboxyl group and side chains of aspartic acid, glutamic acid, cysteine and tyrosine residues); and pKaⱼ is the pKₐ value of the jth acidic group. pKₐ values used are as follows (Nelson, David L., Michael M. Cox, Lehninger Principles of Biochemistry, Fourth Edition):

| Basic Group | pKₐ | Acidic Group | pKₐ |
|---|---|---|---|
| (NH₂—) | 9.69 | (—COOH) | 2.34 |
| K, Lys | 10.5 | D, Asp | 3.86 |
| R, Arg | 12.4 | E, Glu | 4.25 |
| H, His | 6.00 | C, Cys | 8.33 |
| | | Y, Tyr | 10.0 |

In at least one embodiment, the organelle targeting nanocarrier polypeptide includes a chloroplast transit peptide (cTP) sequence and has a net cationic charge equal to or greater than about 2. In at least one embodiment, the polypeptide including a cTP sequence has a net cationic charge of from about 2 to about 6. In at least one embodiment, the polypeptide including a cTP sequence has a net cationic charge of from about 2 to about 4.2.

In at least one embodiment, the organelle targeting nanocarrier polypeptide includes a mitochondrial targeting peptide (mTP) sequence and has a net cationic charge equal to or greater than about 3.5. In at least one embodiment, the polypeptide including a mTP sequence has a net cationic charge of from about 3.5 to about 9.2. In at least one embodiment, the polypeptide including a mTP sequence has a net cationic charge of from about 4 to about 7.

As used herein, the term “hydrophilicity” in reference to a polypeptide is defined as the affinity for water or the tendency to dissolve in, mix with, or be wetted by water. The hydrophilicity of a polypeptide is calculated as the sum of the hydrophilicity values for the individual amino acid residues in the polypeptide, using hydrophilicity values from the Hopp & Woods hydrophilicity scale (Hopp T. P., Woods K. R., Proc. Natl. Acad. Sci. U.S.A. (1981) 78: 3824-3828), as follows:

| | | | | | | | |
|---|---|---|---|---|---|---|---|
| A, Ala | −0.5 | C, Cys | −1 | D, Asp | 3 | E, Glu | 3 |
| F, Phe | −2.5 | G, Gly | 0 | H, His | −0.5 | I, Ile | −1.8 |
| K, Lys | 3 | L, Leu | −1.8 | M, Met | −1.3 | N, Asn | 0.2 |
| P, Pro | 0 | Q, Gln | 0.2 | R, Arg | 3 | S, Ser | 0.3 |
| T, Thr | −0.4 | V, Val | −1.5 | W, Trp | −3.4 | Y, Tyr | −2.3 |

In at least one embodiment, the organelle targeting nanocarrier polypeptide has a hydrophilicity of no more than 0. In at least one embodiment, the polypeptide includes a mitochondrial targeting peptide (mTP) sequence and has a hydrophilicity of from about 0 to about −0.6. In at least one embodiment, the polypeptide including a mTP sequence has a hydrophilicity of about 0 to about −0.5. In at least one embodiment, the polypeptide includes a chloroplast transit peptide (cTP) sequence and has a hydrophilicity of from about 0 to about −0.5. In at least one embodiment, the polypeptide including a cTP sequence has a hydrophilicity of about 0 to about −0.2.

In at least one embodiment, when the polypeptide includes a mitochondrial targeting peptide (mTP) sequence, the polypeptide has a sequence which has at least about 80% similarity, at least about 90% similarity, at least about 95% similarity or at least about 99% similarity to a sequence selected from:

| | |
|---|---|
| MFSYLPRYPLRAASARALVRATRPSYRSALLRYQ; | (SEQ ID NO: 1) |
| MAAWMRSLFSPLKKLWIRMH; | (SEQ ID NO: 2) |
| MKLLWRLILSRKW; | (SEQ ID NO: 3) |
| MWWRRSRTNSLRYT; | (SEQ ID NO: 4) |
| and | |
| MLFRLRRSVRLRGLLA. | (SEQ ID NO: 5) |

In at least one embodiment, when the polypeptide includes a mitochondrial targeting peptide (mTP) sequence, the polypeptide has a sequence which has at least about 80% identity, at least about 90% identity, at least about 95% identity or at least about 99% identity to a sequence selected from:

| | |
|---|---|
| MFSYLPRYPLRAASARALVRATRPSYRSALLRYQ; | (SEQ ID NO: 1) |
| MAAWMRSLFSPLKKLWIRMH; | (SEQ ID NO: 2) |
| MKLLWRLILSRKW; | (SEQ ID NO: 3) |
| MWWRRSRTNSLRYT; | (SEQ ID NO: 4) |
| and | |
| MLFRLRRSVRLRGLLA. | (SEQ ID NO: 5) |

In at least one embodiment, when the polypeptide includes a mitochondrial targeting peptide (mTP) sequence, the polypeptide has a sequence selected from:

| | |
|---|---|
| MFSYLPRYPLRAASARALVRATRPSYRSALLRYQ; | (SEQ ID NO: 1) |
| MAAWMRSLFSPLKKLWIRMH; | (SEQ ID NO: 2) |
| MKLLWRLILSRKW; | (SEQ ID NO: 3) |
| MWWRRSRTNSLRYT; | (SEQ ID NO: 4) |
| and | |
| MLFRLRRSVRLRGLLA; | (SEQ ID NO: 5) |

or an analogous sequence thereof containing one or more deletions, additions or conservative substitutions of amino acid residues, such that the analogous sequence comprises from about 5 to about 35 amino acids.

In at least one embodiment, when the polypeptide includes a chloroplast transit peptide (cTP) sequence, the polypeptide has a sequence which has at least about 80% similarity, at least about 90% similarity, at least about 95% similarity or at least about 99% similarity to a sequence selected from:

| | |
|---|---|
| MGGCVSTPKSCVGAKLR; | (SEQ ID NO: 6) |
| MQTLTASSSVSSIQRHRPHPAGRRSSSVTFS; | (SEQ ID NO: 7) |
| MKNPPSSFASGFGIR; | (SEQ ID NO: 8) |
| MAALIPAIASLPRAQVEKPHPMPVSTRPGLVS; | (SEQ ID NO: 9) |
| and | |
| MSSPPPLFTSCLPASSPSIRRDSTSGSVTSPLR. | (SEQ ID NO: 10) |

In at least one embodiment, when the polypeptide includes a chloroplast transit peptide (cTP) sequence, the polypeptide has a sequence which has at least about 80% identity, at least about 90% identity, at least about 95% identity or at least about 99% identity to a sequence selected from:

| | |
|---|---|
| MGGCVSTPKSCVGAKLR; | (SEQ ID NO: 6) |
| MQTLTASSSVSSIQRHRPHPAGRRSSSVTFS; | (SEQ ID NO: 7) |
| MKNPPSSFASGFGIR; | (SEQ ID NO: 8) |
| MAALIPAIASLPRAQVEKPHPMPVSTRPGLVS; | (SEQ ID NO: 9) |
| and | |
| MSSPPPLFTSCLPASSPSIRRDSTSGSVTSPLR. | (SEQ ID NO: 10) |

In at least one embodiment, when the polypeptide includes a chloroplast transit peptide (cTP) sequence, the polypeptide has a sequence selected from:

| | |
|---|---|
| MGGCVSTPKSCVGAKLR; | (SEQ ID NO: 6) |
| MQTLTASSSVSSIQRHRPHPAGRRSSSVTFS; | (SEQ ID NO: 7) |
| MKNPPSSFASGFGIR; | (SEQ ID NO: 8) |
| MAALIPAIASLPRAQVEKPHPMPVSTRPGLVS; | (SEQ ID NO: 9) |
| and | |
| MSSPPPLFTSCLPASSPSIRRDSTSGSVTSPLR; | (SEQ ID NO: 10) |

or an analogous sequence thereof containing one or more deletions, additions or conservative substitutions of amino acid residues, such that the analogous sequence comprises from about 5 to about 35 amino acids.

As used herein, the term “conservative substitution” is intended to mean a replacement of an amino acid residue in a peptide sequence with a different amino acid residue which has similar chemical and/or physical properties, such that the physical and/or chemical properties of the peptide are only minimally changed by the substitution. Examples of physical and chemical properties include but are not limited to polarity, charge, steric bulk, pKₐ, and aromaticity. For example, one small hydrophobic residue selected from glycine, alanine or valine may be substituted for a different small hydrophobic residue selected from that group; one aromatic residue selected from phenylalanine, tyrosine or tryptophan may be substituted for a different aromatic residue selected from that group; one acidic residue selected from aspartic acid or glutamic acid may be substituted for a different acidic residue selected from that group; one basic residue selected from arginine or lysine may be substituted for a different basic residue selected from that group; one hydroxylated residue selected from serine and threonine may be substituted for a different hydroxylated residue selected from that group, and so forth. The skilled person will be well aware of other amino acid substitutions which are expected to only minimally change the physical and/or chemical properties of the present peptides.

Further aspects of the present invention provide animal cells, plant cells and plants and seeds thereof produced by the methods and compositions described herein. Methods for genetically transforming plant cells, generating plants from genetically transformed plant cells produced by the present methods and generating seed from such plants are well known in the art, including but not limited to biolistic transformation of the nucleus or chloroplasts of plant cells, selection of transformed plant cells using antibiotic resistance markers, and regeneration of whole transgenic plants from transformed isolated microspore cultures (Chugh, A., E. Amundsen, and F. Eudes, Translocation of cell-penetrating peptides and delivery of their cargoes in triticale microspores. Plant Cell Reports (2009) 28(5): 801-810; Lee, S. M., et al., Plastid transformation in the monocotyledonous cereal crop, rice (Oryza sativa) and transmission of transgenes to their progeny. Molecules and Cells (2006) 21(3): 401-410; and Cui, C., et al., Stable chloroplast transformation of immature scutella and inflorescences in wheat (Triticum aestivum L.). Acta Biochimica et Biophysica Sinica (2011) 43(4): 284-291). The skilled person will also have knowledge of other such methods. In addition, methods for genetically transforming the nucleus of animal cells are well known in the art.

### EXAMPLES

The invention as described herein can be more completely understood by reference to the following specific examples, which are presented solely to illustrate specific embodiments of the present invention and are not intended to limit its scope. The skilled person will appreciate that the methods and procedures described herein can be modified, and such modifications are intended to be included. Although specific terms have been used in these examples, such terms are intended to be non-limiting and are used in a descriptive sense. Methods referred to but not explicitly described in the description and in the following examples are well known to persons skilled in the art.

### Example 1

### Identification of Organelle Targeting Peptide (oTP) Sequences

Available protein sequences for wheat (Triticum aestivum), rice (Oryza saliva), maize (Zea mays), and Arabidopsis thaliana were downloaded from NCBI (National Center for Biotechnology Information) GenBank. To eliminate sequence redundancy within the protein sequence datasets, the computer software program Cluster Database at High Identity with Tolerance (CD-HIT) was used to generate a set of non-redundant sequences (Huang, Y., et al., CD-HIT Suite: a web server for clustering and comparing biological sequences, Bioinformatics (2010) 26(5): 680-682). The protein sequences were then analyzed using the TargetP software program to identify N-terminal protein sorting signal sequences and to predict the subcellular localization properties of these N-terminal protein sorting signal sequences (Emanuelsson, O., et al., Predicting Subcellular Localization of Proteins Based on their N-terminal Amino Acid Sequence, Journal of Molecular Biology (2000) 300(4): 1005-1016). N-terminal protein sorting signal peptide sequences specific for chloroplasts (chloroplast transit peptide (cTP) sequences) and mitochondria (mitochondrial targeting peptide (mTP) sequences) were identified from the protein sequence datasets. Candidate signal sequences with potential cell penetrating properties were further selected by the sequential application of specific selection criteria, as summarized in Tables 1 and 2.

TABLE 1
Mitochondrial targeting peptide sequence selection

| | | | | | Net | |
|---|---|---|---|---|---|---|
| | Total starting | | Relative | Sequence | Positive | Average |
| | protein | Total mTP | Confidence | Length | Charge | Hydrophilicity |
| Species | sequences | Sequences | Level ≧0.9 | ≦35 | ≧3.5 | ≦0 |
| Arabidopsis | 387781 | 45588 | 1619 | 503 | 488 | 282 |
| thaliana | | | | | | |
| Rice | 302221 | 55065 | 4635 | 1167 | 701 | 362 |
| (Oryza | | | | | | |
| sativa) | | | | | | |
| Maize | 60709 | 10251 | 423 | 250 | 139 | 81 |
| (Zea mays) | | | | | | |
| Wheat | 34904 | 4594 | 312 | 81 | 23 | 23 |
| (Triticum | | | | | | |
| aestivum) | | | | | | |
| Totals | 785615 | 115498 | 6989 | 2001 | 1351 | 748 |

TABLE 2
Chloroplast transit peptide sequence selection

| | Total | | | | Net | |
|---|---|---|---|---|---|---|
| | starting | | Relative | Sequence | Positive | Average |
| | protein | Total cTP | Confidence | Length | Charge | Hydrophilicity |
| Species | sequences | Sequences | Level ≧0.9 | ≦35 | ≧2.0 | ≦0 |
| Arabidopsis | 387781 | 41132 | 5016 | 503 | 299 | 193 |
| thaliana | | | | | | |
| Rice | 302221 | 40830 | 6571 | 476 | 258 | 115 |
| (Oryza | | | | | | |
| sativa) | | | | | | |
| Maize | 60709 | 9898 | 904 | 78 | 52 | 33 |
| (Zea mays) | | | | | | |
| Wheat | 34904 | 2931 | 373 | 19 | 31 | 8 |
| (Triticum | | | | | | |
| aestivum) | | | | | | |
| Totals | 785615 | 94791 | 12864 | 1076 | 640 | 349 |

The first three columns of numerical data in Tables 1 and 2 represent the total number of starting protein sequences from NCBI GenBank from each organism, the total number of cTP or mTP sequences predicted by TargetP and the predicted number of cTP or mTP sequences with a TargetP relative confidence level of a ≧90%, respectively. The remaining columns of numerical data represent the numbers of sequences predicted when the following selection criteria were applied sequentially and cumulatively: a sequence length of 35 amino acids or less; a net positive charge of a ≧2.0 (for cTPs) or a ≧3.5 (for mTPs); and an average hydrophilicity of ≦0. The charge threshold for mTP sequences was chosen to be higher than that for cTP sequences because mTPs are known to have a relatively high arginine concentration, and therefore a relatively high net positive charge (Bhushan, S., et al., The role of the N-terminal domain of chloroplast targeting peptides in organellar protein import and miss-sorting, FEBS Letters (2006) 580(16): 3966-3972).

### Example 2

### Peptide Cell Penetrating and Organelle Targeting Properties Peptide Synthesis and Labeling

Fifty-four candidate organelle targeting peptide (oTP) sequences (31 mTP sequences and 23 cTP sequences, each selected from those meeting all the criteria outlined above) were synthesized using solid phase Fmoc (fluorenylmethoxycarbonyl) chemistry, as is well known in the art. Each peptide was labelled with fluorescein isothiocyanate (FITC) at the N-terminus, using well known procedures, to facilitate visual detection by fluorescence.

### Triticale Mesophyll Protoplast Isolation and Purification

Protoplast isolation and purification is carried out under aseptic conditions. Embryonal halves of triticale (cv. AC Alta) seeds are surface sterilized with 4% hypochlorite and inoculated on basal MS (Murashige and Skoog) medium, pH 5.82 (Murashige T. and Skoog F. A revised medium for rapid growth and bioassays with tobacco tissue cultures (1962) Physiol. Plant 15(3): 473-497). Peeled leaves from six-day-old seedlings are incubated in enzyme solution [2% cellulase and 2% macerozyme (Yakult Honsha Co Ltd, Japan) in CPW (cell protoplast washing) solution, pH 5.6 (Frearson E M, Power J B, Cocking E C (1973) Dev Biol 33: 130-137)] for 4 hr at 25° C., in the dark. Protoplasts are isolated by centrifugation at 100 g for 3 min, at room temperature (Eppendorf centrifuge 5810R, USA), washed twice with CPW solution and purified by layering on 21% sucrose in CPW solution. A band of protoplasts formed at the interphase is carefully removed and suspended in CPW solution. After two washings with CPW solution, protoplast density is adjusted to 10⁶ protoplasts/ml.

### Tobacco Protoplast Isolation and Purification

Nicotiana benthamiana plants are grown for 6-8 weeks and kept in the dark for 24 hrs prior to all experiments. All pipetting is done slowly with wide bore pipette tips to prevent protoplast lysis. Leaves are cut on the underside in many shallow thin slices, with the center vein taken out, and incubated underside down with enzyme digest solution [2% cellulase and 2% macerozyme (Yakult Honsha Co Ltd, Japan) in CPW (cell protoplast washing) solution, pH 5.6 (Frearson E M, Power J B, Cocking E C (1973) Dev Biol 33: 130-137)] in 15 mm Petri dishes for 3-5 hrs at 28° C. After incubation, leaves are gently shaken with forceps to release any remaining protoplasts, and medium is gently filtered through 100 μM sieves and transferred into 50 mL centrifuge tubes. The medium is spun for 5 min at 300 g at 4° C., and a floating band of protoplasts is removed from the top of the suspension, resuspended in W5 wash solution (154 mM NaCl, 125 mM CaCl₂.2H₂O, 5 mM KCl, 5 mM glucose, 0.5 M mannitol adjusted to pH 5.8 with 0.1 M KOH), and spun for 3 mins at 300 g at 4° C. The pellet is gently layered on 5 mL of 20% maltose in a 15 mL centrifuge tube, and centrifuged 5 mins at 300 g. A floating band in the center of the solution is carefully removed and resuspended with MaMg solution (15 mM MgCl₂, 0.1% MES, 0.4 M mannitol, adjusted to pH 5.6 with KOH), and spun for 3 mins at 300 g at 4° C. The protoplast suspension is diluted to 100,000 cells/ml using a hemocytometer.

### Microspore Isolation and Purification

Isolation of triticale (cv. Alta) microspores at the mid-late uninucleate stage from surface sterilized anthers is carried out in NPB-99 medium, pH 7.0, as described by Eudes et al (Chugh, A., E. Amundsen, and F. Eudes, Translocation of cell-penetrating peptides and delivery of their cargoes in triticale microspores. Plant Cell Reports (2009) 28(5): 801-810).

### MDCK Cell Culture

MDCK cells are cultured in Dulbecco's Modified Eagle Medium (DMEM) containing 5% (v/v) fetal bovine serum (FBS) and 1% (v/v) penicillin/streptomycin at 37° C. in a humidified atmosphere containing 5% CO₂. The cells grown on 10-cm dishes are detached with trypsin and ethylenediaminetetraacetic acid (EDTA) and washed with DMEM/FBS. Cells used for microscopy are prepared by adding 100 000 cells to the apical chamber of 12-mm diameter Transwell™ permeable supports (Costar, Cambridge, Mass.). Cells are cultured in DMEM/FBS for 3-5 days.

### Caco-2 and F1112 Cell Culture

Caco-2 and F1112 cells are cultured in Dulbecco's Modified Eagle Medium (DMEM) containing 10% (v/v) fetal bovine serum (FBS) and 50 μg/ml gentamycin at 37° C. in a 95% humidified atmosphere containing 5% CO₂. The cells are grown in Falcon™ cm² or 75 cm² cell culture flasks until they reach confluency, then are detached with 0.25% trypsin and 0.02% EDTA and washed with DMEM.

Preparations used for photometric cell imaging are prepared by adding 1000 cells to a Nunc coverglass chamber (2.5 cm²). Cells are cultured as described above for 3 to 7 days. The cell monolayers are rinsed three times with epithelial cell saline (pH 7.4).

### MG Cell Culture

MG cells are cultured in Dulbecco's Modified Eagle Medium (DMEM) containing 20% (v/v) fetal bovine serum (FBS) and 50 μg/ml gentamycin at 37° C. in a 95% humidified atmosphere containing 5% CO₂. The cells are grown in Falcon™ 25 cm² or 75 cm² cell culture flasks until they reach confluency, then are detached with 0.25% trypsin and 0.02% EDTA and washed with DMEM.

Preparations used for photometric cell imaging are prepared by adding 1000 cells to a Nunc coverglass chamber (2.5 cm²). Cells are cultured as described above for 3 to 7 days. The cell monolayers are rinsed three times with epithelial cell saline (pH 7.4).

Incubation of Triticale Protoplasts with Fluorescently Labeled Peptides

Mesophyll protoplasts (500 μl of 10⁶/ml preparation) are incubated with 180 μl fluoresceinated mTPs or cTPs (100 μM) for 1 h in the dark at room temperature, followed by washing with CPW solution. Protoplasts are then treated with trypsin-EDTA (0.25%, Sigma-Aldrich) in CPW solution (1:4) for 5 min followed by washing with CPW solution and final suspension in CPW solution (500 μl).

Incubation of Tobacco Protoplasts with Fluorescently Labeled Peptides

A stock solution of fluoresceinated mTPs or cTPs (100 μM) prepared with sterile optimum water is added to protoplast suspension at a final concentration of 20 μM. Each suspension is incubated at room temperature for 1 hour in the dark. Following incubation, 25% Trypsin EDTA is added for 5 minutes and the solution is centrifuged for 2 minutes at 120 g. The pellet is resuspended in fresh MaMg media, and 1.5 μL MitoTracker® Orange CM-H₂TMRos (M7511, Invitrogen) is added for 15 minutes, followed by centrifugation for 2 minutes at 120 g. Protoplasts are mounted on slides in 1% low melting point agar (2:1 ratio of agar to protoplast suspension) for confocal microscopy (Olympus; both XY cross section and XYZ depth images).

Incubation of Microspores with Fluorescently Labeled Peptides

Microspores (500 μl of 10⁶/ml preparation) are incubated with 180 μl fluoresceinated mTPs or cTPs (100 μM) for 1 h in the dark at room temperature, followed by washing with NPB-99 solution. Microspores are then treated with trypsin-EDTA (0.25%, Sigma-Aldrich) in NPB-99 solution (1:4) for 5 min followed by washing with NPB-99 solution and final suspension in NPB-99 solution (500 μl).

Incubation of MDCK Cells with Fluorescently Labeled Peptides

MDCK cells cultured in apical chambers of 12-mm diameter Transwell™ permeable supports are incubated with 80 μl fluoresceinated mTPs or cTPs (100 μM) and 320 μl of Dulbecco's Modified Eagle Medium (DMEM) for 1 h in the dark at room temperature, followed by washing with 400 μl of DMEM.

Incubation of Caco-2, F1112 and MG Cells with Fluorescently Labeled Peptides

Varying concentrations of fluoresceinated mTPs (100 μM) are added to epithelial cell saline to obtain a final working volume of 400 μl. Final concentrations added to the Caco-2, F1112 and MG cell monolayers as described above are 17 μM, 9 μM and 4 μM. Cells are incubated with the peptides for 1 hr in the dark at 37° C. in a 95% humidified atmosphere containing 5% CO₂. At the end of the incubation period, the cell monolayer is rinsed three times with epithelial cell saline and 500 μl of epithelial cell saline is added.

### Confocal Microscopy

Cells are observed using a confocal microscope (Nikon C1, Nikon Canada Inc. or Olympus FluoView™) to analyse the localization of the fluoresceinated mTPs or cTPs (excitation wavelength 490 nm/emission wavelength 520 nm). Chloroplasts are identified by autofluorescence. The fluorescent dye MitoTracker® Orange CM-H₂TMRos (M7511, Invitrogen) is used to stain mitochondria (excitation wavelength 554 nm/emission wavelength 576 nm). Fluorescence emissions are collected in z-confocal planes of 10-15 nm and analyzed using EZ-C1 Software Version 3.6 (Nikon) or Olympus FluoView™ software version 2.0b (Olympus).

### Photometric Cell Imaging

Uptake of labeled peptides by cells is quantified using a photometric detector/cell-imaging system (PTI). An excitation scan is run to determine if uptake has occurred as indicated by peaks at the appropriate wavelengths and if the amount changes in a dose-dependent manner compared to unlabeled cells. A time-based scan is then performed at the optimal excitation and emission wavelength to quantify total uptake, using about 20 cells/view/replicate. The proportion of cells in a view that demonstrate fluorescence is tallied.

### Results

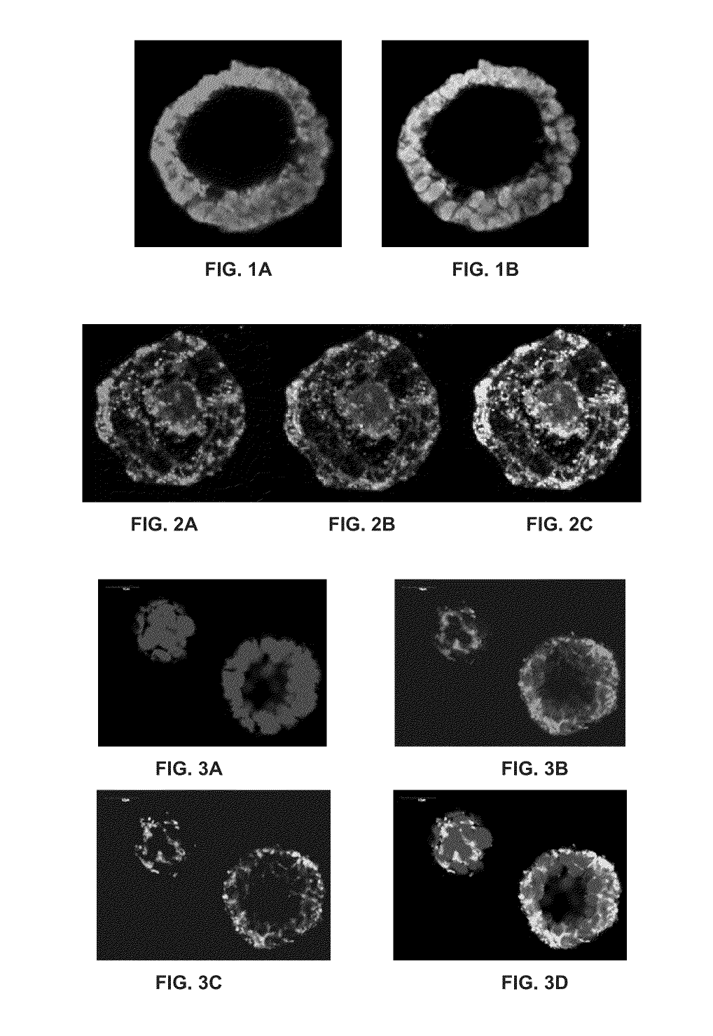

Of the fifty-four candidate peptides synthesized, labeled with fluorescein and incubated with triticale protoplasts, ten organelle targeting peptides (oTPs) were found to both penetrate the cell membrane and localize to either the chloroplasts or the mitochondria, as seen in FIGS. 1A-B and 2A-C. The sequences of these ten peptides are provided in Tables 3 and 4.

TABLE 3
Mitochondrial targeting cell penetrating peptides

| | Peptide | | | |
|---|---|---|---|---|
| mTP | Charge | Hydrophilicity | Peptide sequence | Species |
| mTP1 | 7 | −0.1 | MFSYLPRYPLRAASARALVRATRPSYRSALLRYQ | Wheat |
| | | | (SEQ ID NO: 1) | (Triticum |
| | | | | aestivum) |
| mTP2 | 4.1 | −0.5 | MAAWMRSLFSPLKKLWIRMH (SEQ ID NO: 2) | Maize |
| | | | | (Zea mays) |
| mTP3 | 4 | −0.4 | MKLLWRLILSRKW (SEQ ID NO: 3) | Arabidopsis |
| | | | | thaliana |
| mTP4 | 4 | 0 | MWWRRSRTNLRYT (SEQ ID NO: 4) | Arabidopsis |
| | | | | thaliana |
| mTP5 | 5 | 0 | MLFRLRRSVRLRGLLA (SEQ ID NO: 5) | Arabidopsis |
| | | | | thaliana |

TABLE 4
Chloroplast targeting cell penetrating peptides

| | Peptide | | | |
|---|---|---|---|---|
| cTP | Charge | Hydrophilicity | Peptide sequence | Species |
| cTP1 | 2.9 | 0 | MGGCVSTPKSCVGAKLR (SEQ ID NO: 6) | Arabidopsis |
| | | | | thaliana |
| cTP2 | 4.2 | 0 | MQTLTASSSVSSIQRHRPHPAGRRSSSVTFS | Wheat |
| | | | (SEQ ID NO: 7) | (Triticum |
| | | | | aestivum) |
| cTP3 | 2 | −0.1 | MKNPPSSFASGFGIR (SEQ ID NO: 8) | Rice |
| | | | | (Oryza |
| | | | | sativa) |
| cTP4 | 2.1 | −0.2 | MAALIPAIASLPRAQVEKPHPMPVSTRPGLVS | Wheat |
| | | | (SEQ ID NO: 9) | (Triticum |
| | | | | aestivum) |
| cTP5 | 2 | 0 | MSSPPPLFTSCLPASSPSIRRDSTSGSVTSPLR | Arabidopsis |
| | | | (SEQ ID NO: 10) | thaliana |

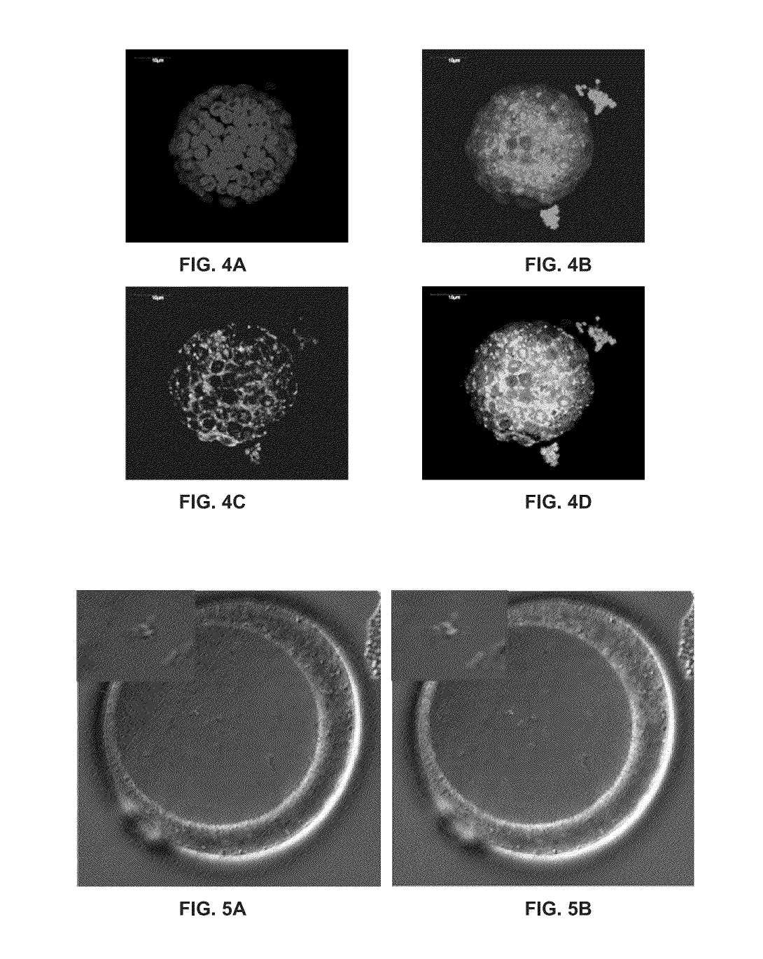

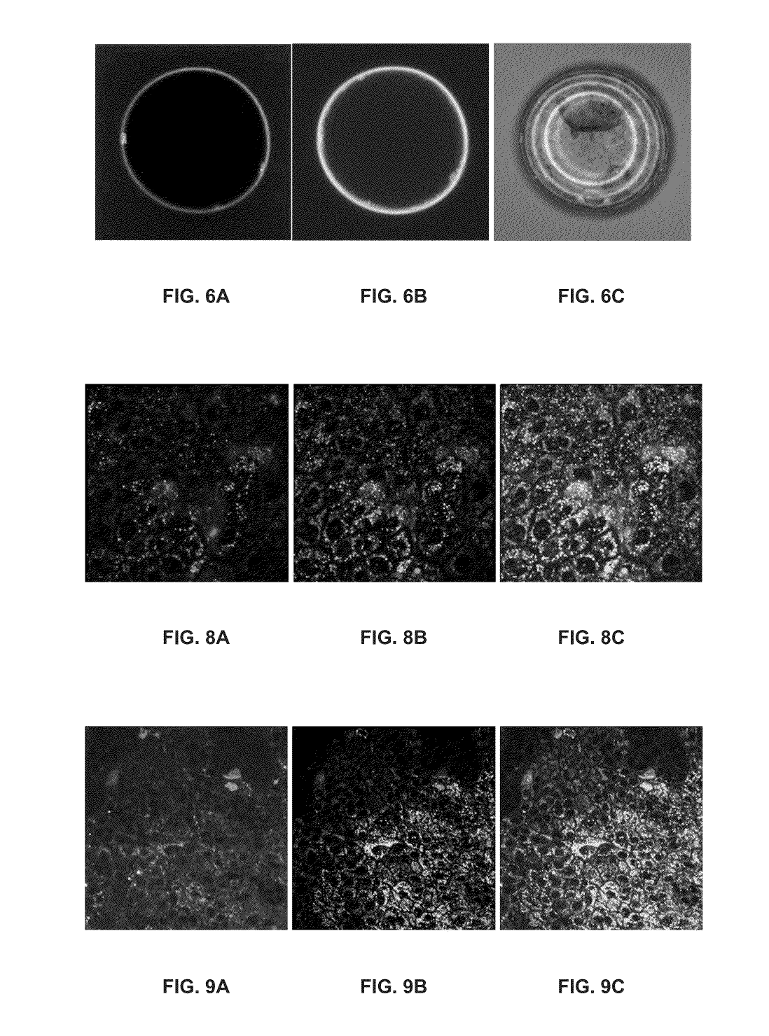

Penetration of the cell membrane and localization to either the chloroplasts or the mitochondria by labelled oTPs was also observed in tobacco protoplasts using confocal microscopy (Olympus). The peptides mTP2 (SEQ ID NO: 2) and mTP3 (SEQ ID NO: 3) were observed to localize to mitochondria (FIGS. 3A-D) and the peptides cTP1 (SEQ ID NO: 6), cTP2 (SEQ ID NO: 7), cTP4 (SEQ ID NO: 9) and cTP5 (SEQ ID NO: 10) were found to localize to chloroplasts (FIGS. 4A-D).

The ten oTPs identified as having both cell penetrating and organelle targeting properties (mTP1-mTP5, Table 3 and cTP1-cTP5, Table 4) were also tested in other cell types, including isolated microspore culture, an alternative plant cell culture system to mesophyll protoplasts, which supports whole plant regeneration. Embryogenic microspores become multicellular and give rise to embryos that regenerate into haploid or double haploid plants (José, M. S.-S, and N. Fernando, How microspores transform into haploid embryos: changes associated with embryogenesis induction and microspore-derived embryogenesis, Physiologia Plantarum (2008) 134(1): 1-12). Transgenic plants can be generated from an isolated microspore culture of wheat and triticale using a nuclear cell-penetrating peptide microspore transfection protocol (Chugh, A., E. Amundsen, and F. Eudes, Translocation of cell-penetrating peptides and delivery of their cargoes in triticale microspores. Plant Cell Reports (2009) 28(5): 801-810).

The ten fluorescein-labelled oTPs identified in Tables 3 and 4 were incubated with triticale microspores and the microspores were observed by confocal microscopy (Nikon) using the procedure described above. It was found that the ten oTPs can penetrate isolated microspores, as seen in FIGS. 5A-B and 6A-C.

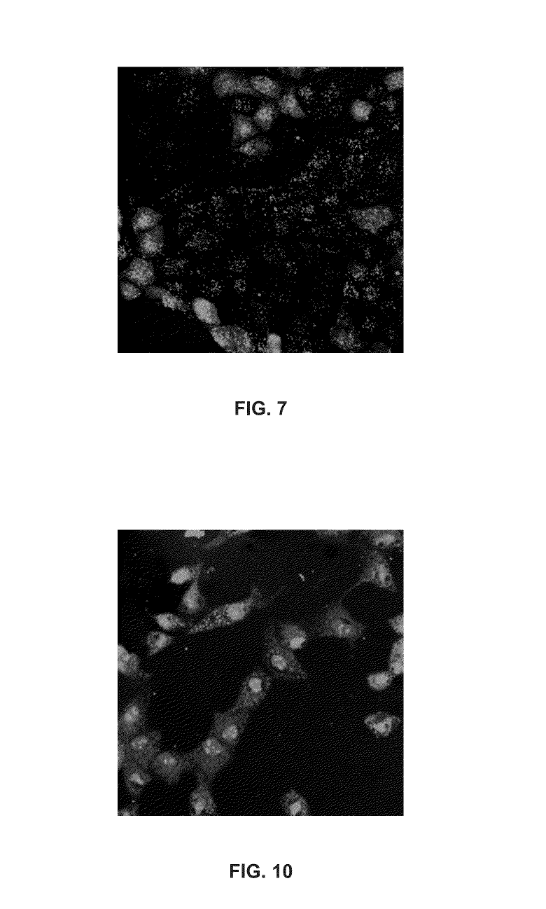

The fluorescein-labelled mTPs listed in Table 3 were also incubated with Madin-Darby canine kidney (MDCK) cells and the cells were observed using confocal microscopy (Nikon) using the procedure described above. As seen in FIGS. 7, 8A-C, 9A-C and 10, all the mTPs tested have cell penetrating properties in MDCK cells. Specific mitochondrial targeting was observed for mTP1 (SEQ ID NO: 1), mTP3 (SEQ ID NO: 3) and mTP5 (SEQ ID NO: 5) (FIGS. 7, 8A-C, 9A-C), while non-specific mitochondrial localization was observed for mTP2 (SEQ ID NO: 2) and mTP4 (SEQ ID NO: 4) (FIG. 10).

The fluorescein-labelled mTPs listed in Table 3 were also incubated with Caco-2 (human colonic epithelial cell line), F1112 (bovine colonic cell line) and MG (bovine mammary gland) cells. Uptake of labeled peptides was measured by photometric cell imaging, using the procedure described above. All cells were fluorescent in the view when an appropriate signal was detected. Table 5 shows the results of uptake of labeled peptides when cells are exposed to varying concentrations (4 μM, 9 μM or 17 μM) of labelled peptides. Background auto-fluorescence for the cell lines is about 1×10⁵ counts/sec. Table 6 shows the average concentration of peptide measured in cells exposed to labeled peptides at a concentration of 17 μM.

TABLE 5
Uptake of varying concentrations of labeled peptides by Caco-2,
F1112 and MG cells

| | | | Photometric Cell Imaging | | |
|---|---|---|---|---|---|
| | Number of | Concentration | (average counts/sec) | | |
| Peptide | replicates | (μM) | Caco-2 | F1112 | MG |
| mTP1 | 3 | 17 | 1600000 | 1200000 | 1900000 |
| | 3 | 9 | 1200000 | 560000 | 1800000 |
| | 3 | 4 | 200000 | 330000 | 400000 |
| mTP2 | 3 | 17 | 490000 | 1700000 | 1500000 |
| | 3 | 9 | 420000 | 1800000 | 160000 |
| | 3 | 4 | 340000 | 450000 | 140000 |
| mTP3 | 3 | 17 | 1600000 | 1300000 | 1500000 |
| | 3 | 9 | 1200000 | 1100000 | 1000000 |
| | 3 | 4 | 460000 | 350000 | 430000 |
| mTP4 | 3 | 17 | 1600000 | 3500000 | 1400000 |
| | 3 | 9 | 1200000 | 300000 | 1200000 |
| | 3 | 4 | 300000 | 250000 | 430000 |
| mTP5 | 3 | 17 | 1600000 | 1600000 | 1600000 |
| | 3 | 9 | 650000 | 2000000 | 1400000 |
| | 3 | 4 | 280000 | 440000 | 440000 |

TABLE 6
Average concentration of labeled peptides measured in Caco-2, F1112
and MG cells exposed to labeled peptides at a concentration of 17 μM

| | Number of | Average concentration (nm) | | |
|---|---|---|---|---|
| Peptide | replicates | Caco-2 | F1112 | MG |
| mTP 1 | 3 | 70 | 49 | 78 |
| mTP 2 | 3 | 20 | 70 | 62 |
| mTP 3 | 3 | 60 | 53 | 62 |
| mTP 4 | 3 | 60 | 144 | 58 |
| mTP 5 | 3 | 60 | 60 | 60 |

### Example 3

### DNA Binding Properties of Peptides

The ten non-labelled oTP sequences identified as having both cell penetrating and organelle targeting properties (Tables 3 and 4) were tested, using a gel mobility shift assay and a nuclease protection assay, for their ability to bind non-covalently to nucleic acids. The results of these assays are shown in Table 7 below.

### Gel Mobility Shift Assay

The gel mobility shift assay is used to determine the minimum peptide concentration needed to bind to plasmid DNA and cause it to shift during electrophoresis. Purified linearized plasmid DNA (100 ng of linear double stranded DNA, 6.8 kb) is mixed with increasing concentrations of each of the ten oTPs listed in Tables 3 and 4, according to calculated increasing peptide:DNA charge ratios (1:1, 2:1, 3:1, 4:1, 5:1, etc.) until a complete shift is observed in the plasmid DNA during electrophoresis. The DNA is prepared to a final concentration of a 100 ng/μl in sterile water. Each reaction has a final volume of 25 μl and is incubated for 30 min for complex formation and subjected to electrophoresis on 1% agarose gel stained with ethidium bromide.

### Nuclease Protection Assay

The ten oTPs listed in Tables 3 and 4 were mixed with plasmid DNA as described for the gel mobility shift assay. For the nuclease protection assay, 5 μl of DNase I (RNase-free DNase set; Qiagen, Valencia, Calif., USA) is added to the mixture volume (50 μl). The mixture is incubated at room temperature for 15 min and then incubated on ice for 5 min. Plasmid-peptide dissociation and plasmid purification is carried out with a commercially available DNA purification kit (QIAquick™ PCR purification kit; Qiagen). DNA is eluted in sterile water. An aliquot of 6 μl is subjected to 1% agarose gel electrophoresis.

TABLE 7
Organelle targeting peptide - DNA binding properties

| | | | Peptide:DNA Ratio | | |
|---|---|---|---|---|---|
| | | | Molar | Highest | |
| | Peptide | Hydro- | Binding | Ratio | Nuclease |
| Peptide | Charge | philicity | Ratio | Tested | Protection |
| cTP1 | 2.9 | 0 | — | 775:1 | + |
| (SEQ ID NO: 6) | | | | | |
| cTP2 | 4.2 | 0 | — | 975:1 | + |
| (SEQ ID NO: 7) | | | | | |
| cTP3 | 2 | −0.1 | — | 1400:1 | − |
| (SEQ ID NO: 8) | | | | | |
| cTP4 | 2.1 | −0.2 | — | 2050:1 | − |
| (SEQ ID NO: 9) | | | | | |
| cTP5 | 2 | 0 | — | 1775:1 | − |
| (SEQ ID NO: 10) | | | | | |
| mTP1 | 7 | −0.1 | 264.14 | 800:1 | + |
| (SEQ ID NO: 1) | | | | | |
| mTP2 | 4.1 | −0.5 | 167.39 | 800:1 | + |
| (SEQ ID NO: 2) | | | | | |
| mTP3 | 4 | −0.4 | 71.39 | 2650:1 | + |
| (SEQ ID NO: 3) | | | | | |
| mTP4 | 4 | 0 | — | 2700:1 | + |
| (SEQ ID NO: 4) | | | | | |
| mTP5 | 5 | 0 | 114.29 | 2050:1 | + |
| (SEQ ID NO: 5) | | | | | |

### Results

As seen in Table 7, four of the five mTP peptides caused a mobility shift in the DNA during electrophoresis, indicating that binding to DNA was occurring. The remaining oTPs listed in Table 7 did not show a mobility shift at the peptide:DNA ratios tested. Furthermore, the results of the nuclease protection assay show that organelle targeting peptides having a cationic charge of ≧2.9 protected the DNA from nuclease degradation. This data indicates that oTPs with a cationic charge of ≧2.9 possess the ability to bind to DNA in a non-covalent manner, suggesting that such peptides could be utilized to deliver nucleic acids to specific plant cell organelles.

### Example 4

### DNA Delivery Properties of Peptides

The ten non-labelled oTPs listed in Tables 3 and 4 were tested for their capability to deliver biologically active DNA to chloroplasts or mitochondria using an oTP-GFP reporter construct transfection assay. oTPs are mixed with a double stranded (ds) DNA construct encoding Aequorea victoria green fluorescent protein (GFP) to form a complex. The plasmid-peptide nanocomplexes formed are incubated with triticale mesophyll protoplasts, microspores, or MDCK, Caco-2, F1112 or MG cells. Detection of a fluorescent signal by confocal microscopy and/or photometric cell imaging would indicate that the dsDNA had been transported into organelles and that transient expression of GFP had occurred. In addition, gene expression can be measured using quantitative real-time PCR to determine gfp mRNA abundance.

dsDNA Construct for Expression in Mitochondria

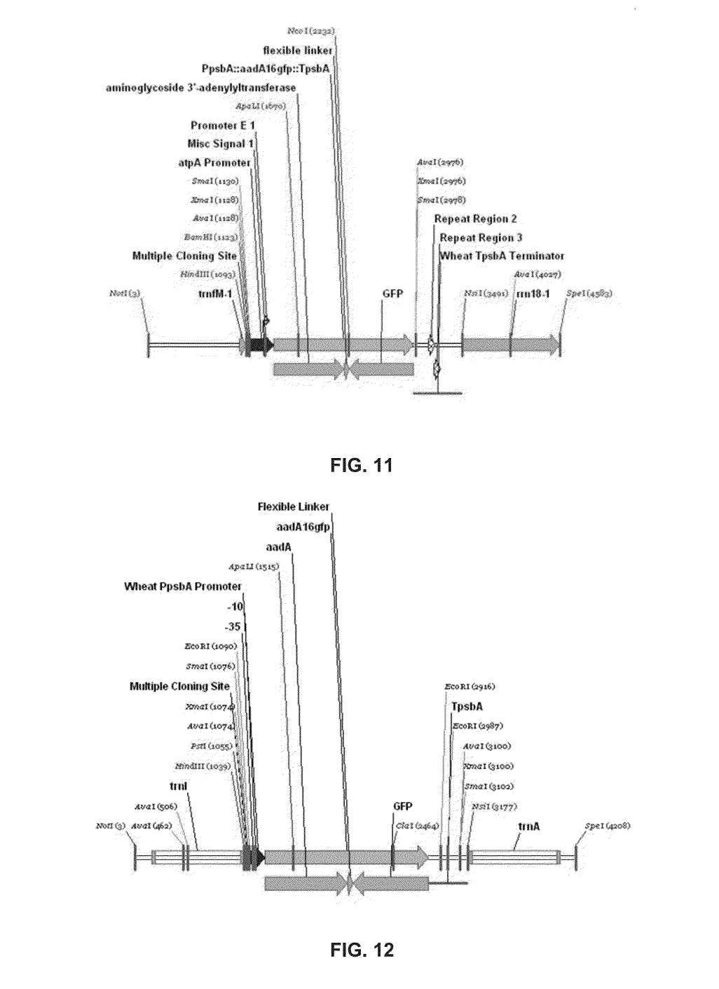

Wheat mitochondrial aadA16GFP reporter plasmid (pWMaadA16GFP, FIG. 11) is a 4587 base pair wheat specific mitochondrial transformation vector, which targets insertions into the fourth repeat region between the trnfM and rm18 gene cluster which is repeated three times in the Triticum aestivum mitochondrial genome. The insertion site is in the Triticum aestivum mitochondrial genome at nucleotides 300805-300878 and 300880-302834 (GenBank accession No. AP008982.1). A multiple cloning site is introduced following the tmfM target insertion sequence. The selection marker gene is an organelle codon specific aad-gfp fusion gene, which facilitates a dual selection method of spectinomycin resistance through an aadA derivative and visual detection by GFP fluorescence (GenBank accession No. ABX39486; Khan and Maliga, Nat. Biotechnol. (1999 September) 17(9): 910-5). The selection marker gene is driven by the Triticum aestivum mitochondrial atpA gene promoter (GenBank accession No. X54387.1). The aad-gfp fusion gene is terminated with the TpsbA terminator sequence derived from the Triticum aestivum mitochondrial genome at nucleotides 62871-62565 (GenBank accession No. AP008982.1).

dsDNA Construct for Expression in Chloroplasts

Wheat chloroplast aadA16GFP reporter plasmid (pWCaadA16GFP, FIG. 12) is a 4212 base pair wheat specific plastid transformation vector, which targets insertions into the tml-tmA inverted repeat regions of the Triticum aestivum plastid genome at nucleotides 92850-93727 and 93794-94671 (GenBank accession No. AB042240.3). A multiple cloning site is introduced following the tml target insertion sequence. The selection marker gene is an organelle codon specific aad-gfp fusion gene, which facilitates a dual selection method of spectinomycin resistance through an aadA derivative and visual detection by GFP fluorescence (GenBank accession No. ABX39486; Khan and Maliga, Nat. Biotechnol. (1999 September) 17(9): 910-5). The selection marker gene is driven by the Triticum aestivum plastid genome promoter (psbA) at nucleotides 1282-1153 (GenBank accession No. AB042240.3). The aad-gfp fusion gene is terminated with the rice psbA terminator sequence derived from chloroplast transformation vector pVSR326 nucleotides 4014-4387 (GenBank accession No. AF527485.1).

Protoplast Transformation with gfp Reporter

The dsDNA construct (pWMaadA16GFP for mitochondrial expression or pWCaadA16GFP for expression in chloroplasts) is combined with the mTP (for mitochondrial expression) or cTP (for expression in chloroplasts) in a final volume of 100 μl of CPW solution. For experiments using mTP1 (SEQ ID NO: 1), mTP2 (SEQ ID NO: 2), mTP3 (SEQ ID NO: 3), or mTP5 (SEQ ID NO: 5), peptides (scaled up from 100 ng, at four times the concentration needed to cause a DNA shift in the gel mobility shift assay (Table 7)) are combined with the pWMaadA16GFP construct (5 μg). For experiments using the remaining oTPs, peptides (30 μg) are combined with the appropriate dsDNA construct (1.5 μg). The mixture is incubated for 10 minutes at room temperature, then incubated with isolated triticale mesophyll protoplasts (500 μl, 10⁶ protoplasts/ml, prepared as described in Example 2) for 1 h in the dark at room temperature. CPW solution (500 μl) is added and the mixture is incubated in the dark for 24 h. The cells are imaged using confocal microscopy, as described in Example 2, using MitoTracker® Orange staining to visualize mitochondria and chlorophyll autofluorescence to visualize chloroplasts.

Microspore Transformation with gfp Reporter

The dsDNA construct (pWMaadA16GFP for mitochondrial expression or pWCaadA16GFP for expression in chloroplasts) is combined with the mTP (for mitochondrial expression) or cTP (for expression in chloroplasts) in the amounts used in the protoplast transformation experiments described above in a final volume of 100 μl of NBP-99 solution. The mixture is incubated for 10 minutes at room temperature, then incubated with isolated triticale microspores (500 μl, 10⁶ microspores/ml, prepared as described in Example 2) for 1 h in the dark at room temperature. NBP-99 solution (500 μl) is added and the mixture is incubated in the dark for 24 h. The cells are imaged using confocal microscopy, as described in Example 2, using MitoTracker® Orange staining to visualize mitochondria.

MDCK Cell Transformation with gfp Reporter

The dsDNA construct (pWMaadA16GFP) is combined with the mTP in the amounts used in the protoplast transformation experiments described above in a final volume of 100 μl of Dulbecco's Modified Eagle Medium (DMEM). The mixture is incubated for 10 minutes at room temperature, then incubated with MDCK cells (prepared as described in Example 2) in 300 μl of DMEM at 37° C. in a humidified atmosphere containing 5% CO₂ for 24 h. The cells are imaged using confocal microscopy, as described in Example 2, using MitoTracker® Orange staining to visualize mitochondria.

Caco-2, F1112 and MG Cell Transformation with gfp Reporter

The dsDNA construct (pWMaadA16GFP. 100 μl of the stock solution, 5 μg) is combined with mTP1 (SEQ ID NO: 1) or mTP4 (SEQ ID NO: 4) (100 μl of the unlabelled stock, 500 μg) and incubated for 15 minutes at 37° C. An 800 μl aliquot of DMEM is added to the mixture and 100 μl of this mixture is then added to each cell monolayer (prepared as described in Example 2) that has 500 μl complete media. The cells are incubated at 37° C. in a humidified atmosphere containing 5% CO₂ for 24 h to 72 hr. The uptake and expression of the GFP reporter is measured at 40 h using a photometric detector/cell-imaging system (PTI) as described in Example 2. The cells are also imaged using confocal microscopy as described in Example 2 using MitoTracker® Orange staining to visualize mitochondria.

RNA Extraction from Transfected Microspores

Microspores are disrupted using a cleaned ceramic bead and aggressive shaking as the tissue is thawing. RLT buffer/B-Me (450 μl) is added and the sample is vortexed. The sample is heated at 55° C. for 1 minute and vortexed again. RNA is extracted using an RNeasy™ Plant Mini Kit (Qiagen), incorporating on-column DNase I digestion into the protocol. The final elution volume is 40 μl. RNA is assessed using both agarose gel chromatography (to ensure RNA is intact) and spectrophotometry (for quantification).

cDNA Synthesis

cDNA is generated using a First Strand Synthesis kit (Invitrogen). RNA (1 μg) is added to each sample and the manufacturer's protocol for synthesis followed, with the exception that the volume of the reaction mixture is increased from 20 μl to 30 μl. A negRT sample containing all reagents but no RNA is included in the cDNA synthesis as a negative control.

### Real Time PCR

Standard curves are generated for both elongation factor 1a (EF1a) (internal control) and green fluorescent protein (GFP) (test) genes. The EF1a curve is generated by pooling 6 μl of each cDNA sample (excluding the negRT control), then carrying out a serial dilution of 0.5 for 5 more dilutions. The GFP standard curve is generated using the plasmid DNA that is used for transfection (13 ng/μl plasmid, of which 3 μl (40 ng) are used for the PCR reaction, followed by a dilution series of 0.5 for 5 more samples).

Real time PCR reactions are performed using QuantiTect™ SYBR™ Green PCR Master Mix (Qiagen) in a 20 μl reaction volume. Each sample is reacted in triplicate and 3 μl of template are used for each reaction. Cycling is performed as follows: 95° C. for 15 min, 40 repeats of 94° C. for 15 sec, 58° C. for 30 sec, 72° C. for 30 sec. Dissociation curves are run after the PCR reactions are complete. The three data points are averaged for each sample and a standard deviation calculated. Standard curves are generated by plotting average C_{T} value against the log of the amount of DNA in each sample so that the PCR efficiency of each primer set could be assessed. The primer sets are amplified with comparable efficiency so C_{T} values can be directly compared.

### Results

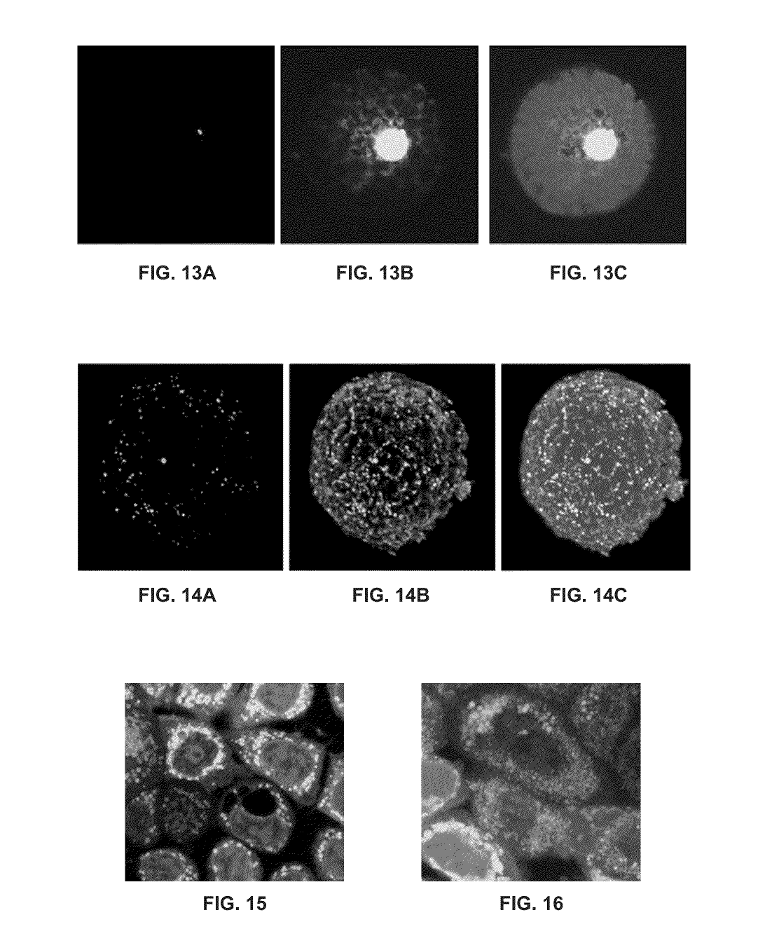

As seen in FIGS. 13A-C and 14A-C, mTP4 (SEQ ID NO: 4) and mTP2 (SEQ ID NO: 2) can mediate the transfection of mitochondria of triticale mesophyll protoplasts. Mitochondria were observed to be fluorescing green by confocal microscopy, indicating that the mitochondria in the mTP transfected protoplasts were expressing GFP. Expression of GFP was also detected in mitochondria of Caco-2, F1112 and MG cells transfected with the mitochondrial gfp-reporter construct in the presence of mTP1 (SEQ ID NO: 1) and mTP4 (SEQ ID NO: 4) by photometric cell imaging, carried out as described in Example 2. However, as seen in Table 8, the signal strength observed upon transfection in the presence of mTP4 (SEQ ID NO: 4) was weak and no fluorescence was observed. In contrast, the signal strength observed upon transfection in the presence of mTP1 (SEQ ID NO: 1) was higher than that observed for mTP4 (SEQ ID NO: 4) and fluorescence was observed in all cells. Confocal imaging of transfected Caco-2 and F1112 cells confirmed the localization of gfp expression to the mitochondria (FIGS. 15 and 16).

TABLE 8
Detection of gfp expression in Caco-2, F1112 and MG cell lines

| | | Photometric Cell Imaging | | |
|---|---|---|---|---|
| | Number of | (average counts/sec) | | |
| Peptide | replicates | Caco-2 | F1112 | MG |
| mTP1 (SEQ ID NO: 1) | 3 | 2000000 | 1900000 | 1900000 |
| mTP4 (SEQ ID NO: 4) | 3 | 570000 | 650000 | 650000 |

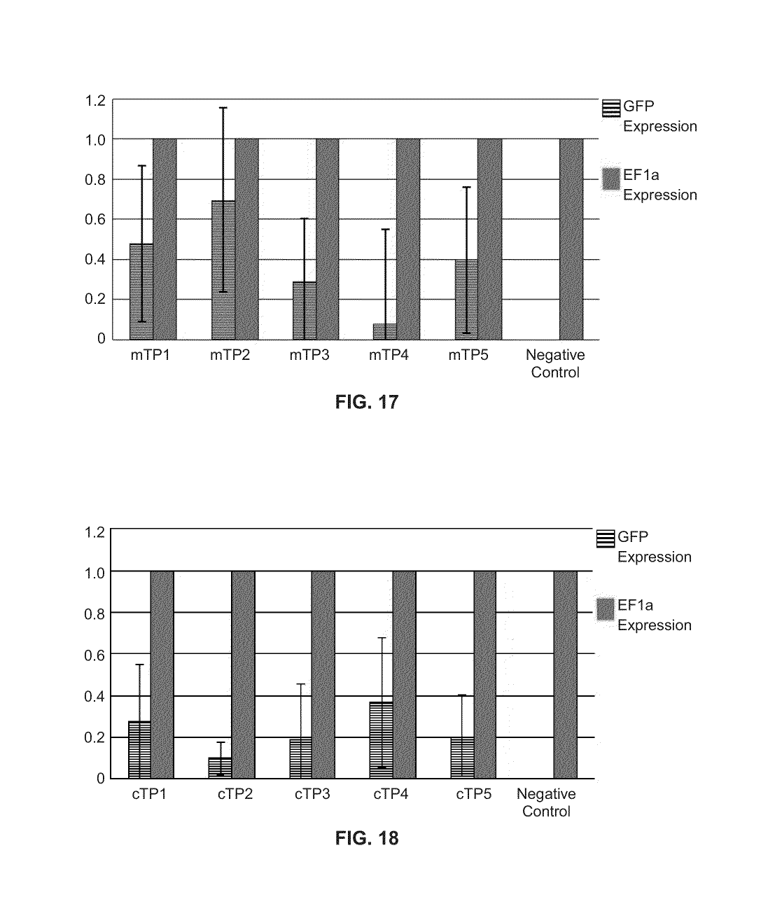

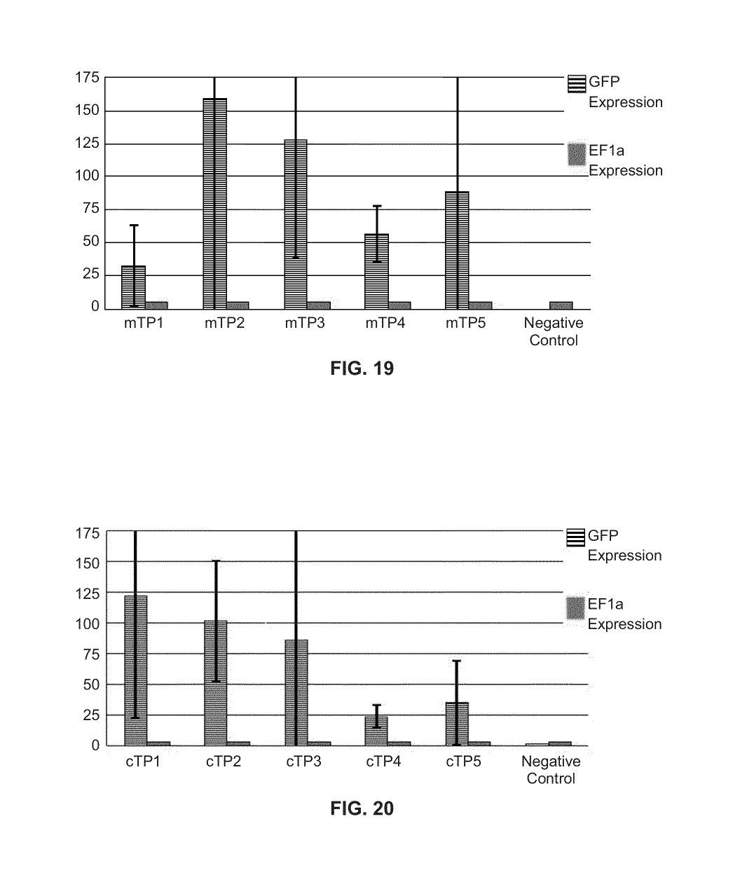

In addition, quantitative real-time PCR (qRT-PCR) analysis confirmed that GFP is expressed by mitochondria of microspores and protoplasts transfected with a gfp reporter construct in the presence of mTP1 (SEQ ID NO: 1), mTP2 (SEQ ID NO: 2), mTP3 (SEQ ID NO: 3), mTP4 (SEQ ID NO: 4) or mTP5 (SEQ ID NO: 5). Normalized gfp mRNA abundance from transfected mitochondria showed a 0.1-0.7 fold increase (average of 4 replicate experiments) in microspores (FIG. 17) and a 32-159 fold increase (average of 4 replicate experiments) in protoplasts (FIG. 19) compared to abundance of the internal control elongation factor 1a (EF1a) mRNA. Furthermore, quantitative real-time PCR (qRT-PCR) analysis confirmed that GFP is expressed by proplastids of microspores, and chloroplasts of protoplasts, each transfected with a gfp reporter construct in the presence of cTP1 (SEQ ID NO: 6), cTP2 (SEQ ID NO: 7), cTP3 (SEQ ID NO: 8), cTP4 (SEQ ID NO: 9) or cTP5 (SEQ ID NO: 10). Normalized gfp mRNA abundance from transfected proplastids showed a 0.10-0.37 fold increase (average of 4 replicate experiments) in microspores (FIG. 18), and normalized gfp mRNA abundance from transfected chloroplasts showed a 24-122 fold increase (average of 4 replicate experiments) in protoplasts (FIG. 20), compared to abundance of the internal control elongation factor 1a (EF1a) mRNA.

### Example 5

### Propagation of Plants from Transformed Microspores

Microspores isolated from triticale cultivar Ultima were transfected with the mitochondrial reporter plasmid WMaadAGFP (Example 4) in the presence of mTP1 (SEQ ID NO: 1), or with the chloroplast reporter plasmid WCaadAGFP (Example 4) in the presence of cTP1 (SEQ ID NO: 6). Plants were regenerated from the transformed microspores and grown under spectinomycin selection, and were characterized by quantitative PCR to measure copy number of green fluorescent protein (GFP) DNA in transfected mitochondria or chloroplasts. Plants which were positive for aadA-gfp transfection were cultured in soil without spectinomycin selection pressure to test for reversion to wild type. Characteristics of the plants produced are shown in Table 9 below.

### Microspore Isolation

Awns of triticale cultivar Ultima are removed with scissors in a laminar flow hood. Spikes (eight spikes for microspore isolation and four spikes to supply ovaries) are sterilized with 10% bleach (5.25% sodium hypochlorite) for 3 min and rinsed four times for 1 min with sterile double distilled water with constant agitation. Outer glumes are removed and florettes from eight spikes are aseptically dissected and transferred to a sterile and refrigerated 110 ml Waring blender cup (VWR international, #58983-093) containing 50 ml filter sterilized extraction solution (0.4 M mannitol, GEM (Germination of Embryo of Monocots) macrosalts (F. Eudes, S. Acharya, A. Laroche, L. B. Selinger & K.-J. Cheng. A novel method to induce direct somatic embryogenesis, secondary embryogenesis and regeneration of fertile green cereal plants. Plant Cell, Tissue and Organ Culture (2003) 73: 147-157), 10 mM 2-(N-morpholino)ethanesulfonic acid (MES), and 100 mM Fe-EDTA, pH 6.5) liquid medium at 4° C. Florettes are blended twice for 7 s at low speed (18000 rpm). The suspension is poured through a 1 mm sieve and then 100 μm sterile mesh (VWR International, #CA21008-950) into two 50 ml centrifuge tubes (25 ml each). The blender cup is rinsed with 50 ml extraction solution at 4° C. and poured through the 100 μm mesh and added to the first aliquot in the 50 ml tubes. The cells are then pelleted by centrifugation (100×g for 5 min at 4° C.) using a swinging bucket rotor. The supernatant is poured off and the microspore pellets are combined in one 50 ml tube and resuspended in 50 ml cold extraction solution. The cells are pelleted again with centrifugation (100×g for 5 min at 4° C.), the supernatant is poured off and the pellet is transferred (in approximately 5 ml) to a 15 ml tube. The pellet is resuspended in 15 ml induction medium (NPB-99 solution supplemented with 2 μM glutathione and 10 mg/l Larcoll™ (arabinogalactan)) and is washed and centrifuged under the same conditions as above. The supernatant is poured off and the pellet is resuspended in 5 to 6 ml of 20% maltose, then 1 mL of induction medium is carefully layered on top of the maltose and the tube is centrifuged at 100 g for 13 min (maltose gradient purification). A band of microspore forms at the interface and is collected in a new 15 ml tube. The tube is filled with induction medium and centrifuged again at 150 g for 5 min. The supernatant is poured off and the cells are suspended in a total volume of 1.4 ml. For each microspore extraction and purification, the concentration of cells is determined using a hemocytometer. Each microspore extraction allowed generation of 15 to 20 experimental units.

Preparation of dsDNA-oTP Complexes:

A midi-prep is carried out with plasmids pWMaadA16GFP and pWCaadA16GFP (Example 4) for organelle transfection. pWMaadA16GFP is double digested using AvrII and SpeI restriction enzymes, and pWCaadA16GFP is double digested using AatII and XmnI restriction enzymes, according to NEB (New England Biolabs) instructions. The genetic cassette (dsDNA) is gel purified.

For mitochondria transfection, 1.5 μg of pWMaadA16GFP dsDNA and 7.5 μg mTP1 (SEQ ID NO: 1) are mixed together in 100 μL in a 1.5 mL microcentrifuge tube. For chloroplast transfection, 1.5 μg of pWCaadA16GFP dsDNA and 30 μg cTP1 (SEQ ID NO: 6) are mixed together in 200 μL in a 1.5 mL microcentrifuge tube. Complexes are incubated for 15 min at RT before use.

### Transfection of Microspores

The dsDNA-oTP complexes (100 or 200 μl) are added to the microspores, gently mixed, and incubated with the complexes for 15 min. 100 μl of induction medium (NPB-99 solution supplemented with 2 μM glutathione and 10 mg/l Larcoll™ (arabinogalactan)) is added, and the mixture is incubated for 45 min at RT. The transfected microspores are washed once with induction medium, centrifuged and the supernatant removed. A control treatment, containing no DNA and nanocarrier, is applied to two experimental units from each batch of purified microspores. Microspore culture is resumed as described in F. Eudes and E. Amundsen. Isolated microspore culture of Canadian 6 triticale cultivars. Plant Cell. Tissue and Organ Culture (2005) 82: 233-241.

### Regeneration of Transformed Plants

Transfected microspores are pipetted (0.2 ml) into 35×10 mm Petri dishes each containing 3.3 ml induction medium with 10% Ficoll™. Four or five ovaries from sterilized spikes taken directly from triticale cultivar Ultima plants are added to each dish containing microspores. The dishes are sealed with Parafilm™ and placed in a 150 mm Petri dish around an open 50 mm Petri dish containing sterile distilled water. The 150 mm dish is also sealed with Parafilm™ and incubated in the dark at 25° C. for 20 to 30 days. Embryos larger than 0.5 mm are removed from the Petri dishes and plated onto GEM medium (20 ml in 10 cm Petri dishes) (F. Eudes, S. Acharya. A. Laroche, L. B. Selinger & K.-J. Cheng. A novel method to induce direct somatic embryogenesis, secondary embryogenesis and regeneration of fertile green cereal plants. Plant Cell, Tissue and Organ Culture (2003) 73: 147-157). The Petri dishes are again sealed with Parafilm™ and placed 30 cm beneath Sylvania Gro-lux™ wide spectrum bulbs (40 watts) delivering 80 μM m⁻² s⁻¹ (16 h light period) at a room temperature of 16° C. Once the embryos turn green, they are aseptically transferred onto 50 ml rooting media (F. Eudes, S. Acharya, A. Laroche, L. B. Selinger & K.-J. Cheng. A novel method to induce direct somatic embryogenesis, secondary embryogenesis and regeneration of fertile green cereal plants. Plant Cell, Tissue and Organ Culture (2003) 73: 147-157) in Magenta™ Vessels (VWR International), in the same conditions. Once the plants reach a 2-3 leaf stage and have sufficient root growth, they are transplanted into soil (4×8 Spencer-Lemaire Rootrainer™; Spencer-Lemaire Industries Ltd., Edmonton) and placed into a growth cabinet under the same conditions as the mother plants. Two weeks after anthesis, ploidy level is estimated by checking for seed set.

### Antibiotic Selection

Spectinomycin selection is applied to selected batches of the microspore culture and to green plants grown in soil. In Batch 1, developing embryos are transferred to a RITA™ box semi-automated immersion culture system at 3 weeks using 200 ml of liquid GEM culture medium supplemented with 200 μl PPM™, and 200 or 400 mg/L spectinomycin is added. In Batch 2, microspores are subjected to a first dose of 100 mg/L spectinomycin at the start of culture. At 3-4 weeks, developing multicellular structures are transferred into a RITA™ box semi-automated culture system, and spectinomycin concentration is increased to 200 mg/L. Two weeks later, the culture medium is replaced with fresh liquid GEM supplemented with 200 μl PPM™ (200 ml), and a third dose of spectinomycin is applied, at 400 mg/L. After two more weeks at high selection pressure, germinated (green and variegated) plantlets are transferred to Rootrainers™. The spectinomycin selective pressure is not applied in soil for plants derived from batches 1 and 2.

In subsequent batches, microspores are subjected to a first dose of 50 mg/L spectinomycin at the start of culture. At 3-4 weeks, developing multicellular structures are transferred into a RITA™ box semi-automated culture system, and spectinomycin concentration is increased to 100 mg/L. Two weeks later, the culture medium is replaced with fresh liquid GEM supplemented with 200 μl PPM™ (200 ml), and a third dose of spectinomycin is added 200 mg/L). After two more weeks, germinated (green and variegated) plantlets are transferred to Rootrainers™. The spectinomycin selective pressure is then applied in soil at concentration of 400 mg/L spectinomycin. Plants cultured in soil are continuously watered from the bottom with a 400 mg/L spectinomycin solution.

Genomic DNA and RNA Extraction from Regenerated Green Plants

Leaf samples are disrupted using a cleaned ceramic bead and aggressive shaking as the tissue is thawing. RLT buffer/B-Me (450 μl) is added and the sample is vortexed. The sample is heated at 55° C. for 1 minute and vortexed again. RNA is extracted using an AllPrep™ DNA/RNA Mini Kit (50) (Qiagen), incorporating on-column DNase I digestion into the protocol. The final elution volume is 40 μl. RNA is assessed using both agarose gel chromatography (to ensure RNA is intact) and spectrophotometry (for quantification of DNA and RNA).

SYBR™ Green qPCR Assay for Determination of Copy Number

The SYBR™ Green real time PCR assay is carried out as described in Example 4. C_{T} values determined are fitted to the standard curve for that primer set and copy number is calculated relative to input DNA using the curve formula. Standard curves are made by diluting mitochondrial or chloroplast reporter plasmids (Example 4) in a series of 6 samples of 1/10 dilutions. Copies in the standard curve for the mitochondrial reporter plasmid ranged from 882,352/μl to 0.8/μl. Copies in the standard curve for the chloroplast reporter plasmid ranged from 234,042/μl to 0.2/μl. All genomic DNA samples are quantitated via spectrophotometer, and samples for real time are prepared so that all reactions contain 2×SYBR™ Green QuantiTect™ Master Mix (Qiagen) (12.5 μl), Gfp4L Fwd primer (10 μM, 1 μl), Gfp4R Rev primer (10 μM, 1 μl), and DNA (200 ng, 11 μl). All biological samples are run in triplicate. Cycling is performed as follows: 95° C. for 15 min, 35 repeats of 95° C. for 15 sec, 60° C. for 30 sec, 72° C. for 30 sec. Resulting C_{T} values are averaged. Standard deviation is calculated for each triplicate of samples and outliers are discarded. Standard curves are generated by plotting average C_{T} value against the log of the amount of DNA in each sample. Average C_{T} value is fitted to the formula generated by the regression curve of each plasmid (y=mx+b). For the mitochondrial reporter standard curve, the equation is y=−3.4845x+43.742, and the value of R² is 0.992. For the chloroplast standard curve, the equation is y=−3.256x+45.469, and the value of R² is 0.9705. The resulting log value is then converted to actual copies and this value is compared to the total number of copies of background DNA in each 200 ng sample. Copies in 200 ng are calculated using the formula:

Number of bp Ultima (19,000 Mb)×660 g/mole (weight of a bp)×109 ng/g.

Taq Man qPCR Assay for Determination of Copy Number

Real time PCR reactions are performed in 96-well plates using the 7900HT Fast Real Time PCR system (Applied Biosystems) and the Qiagen chemistry. The TaqMan probe for the gfp gene is labeled at the 5′ end with FAM™; the probe for the pKABA1 gene is labeled at the 5′ end with VIC™. Both probes are labeled with tetramethylrhodamine (TAMRA™) at the 3′ end as the quencher molecule. For each reaction, 2 μl of DNA, 12.5 μl 2× TaqMan Universal PCR Master Mix (Applied Biosystem, Foster City, Calif.), 0.4 μM gfp and PKABA1 primers and 200 nM of each dual-labeled probe are added, and reactions are brought to a final volume of 25 μl with H₂O. PCR is performed as follows: 10 min at 95° C., 40 cycles of 1 min at 95° C., 1 min at 58° C. All reactions are run in duplicate and repeated on two biological samples. Copy number is calculated using the formula 2^{-ΔΔC}_{T}. An adjustment to the ΔC_{T} value of the calibrator sample of −1 reflects the fact that the endogenous control gene (PKABA) is represented only by the contribution of the wheat genomes A and B to triticale. The rye genome R does not contain this gene (2/3 chromosome sets of a haploid genome have the PKABA gene). The standard curve is also established by serial dilution of the target template and genomic DNA, as well as target gene dilution in genomic DNA. The PCR efficiency of each primer set, the dynamic range of the primers and presence or absence of primer competition in the multiplex reactions are determined. A calibrator sample is also included in each qPCR run.

### Results

A list of plants produced from transfected microspores is provided in Table 9. Green, albino and variegated plants were produced. In Batch 1, in which embryos were not exposed to spectinomycin until 3 weeks after fertilization, the variegated phenotype could be observed in the regenerated plants in greater abundance. Subsequent batches mainly produced a mix of albino and green plants. Haploid and doubled haploid triticale plants from batches 1 and 2 were characterized by qPCR at the time of initial transfer to soil (Rootrainers™). A few triticale lines from batches 1 and 2 were identified by qPCR (using either the SYBR™ Green method or the Taq Man method) as having a positive indication of integration of the reporter gene in the organelle genome. Plants from batch 2 were cultured in soil in the absence of spectinomycin selection pressure, and were found to have reverted back to wild type after a month, as indicated by the reduction in copy number measured by qPCR and evidence that the cytoplasm remained heteroplastomic at that time.

TABLE 9
Triticale haploid plants produced by transfection of microspores

| | | | Real-time PCR screening | | | |
|---|---|---|---|---|---|---|
| | | | (gfp copies/genome) | | | Phenotype |
| | | Organelle | | | Taq Man 2 | after 1-2 |
| | | targeted for | SYBR ™ | Taq | (1 month | months in |
| Plant Name | Batch | transfection | Green | Man 1 | in soil) | soil |
| control with 400 | 1 | — | 0 | | | dead |
| selection-01 | | | | | | |
| control with 400 | 1 | — | 0 | | | Variegated |
| selection-02 | | | | | | |
| control with 400 | 1 | — | 0.5 | | | dead |
| selection-03 | | | | | | |
| control with 400 | 1 | — | 0.2 | | | dead |
| selection-04 | | | | | | |
| control with 400 | 1 | — | 0 | | | Variegated |
| selection-05 | | | | | | |
| control with 400 | 1 | — | 0 | | | dead |
| selection-06 | | | | | | |
| control with 400 | 1 | — | 0 | | | dead |
| selection-07 | | | | | | |
| control with 400 | 1 | — | 0 | | | Variegated |
| selection-08 | | | | | | |
| control without | 1 | — | 0.6 | | | Variegated |
| selection-01 | | | | | | |
| control without | 1 | — | 0.2 | | | Variegated |
| selection-02 | | | | | | |
| control without | 1 | — | 0.3 | | | Variegated |
| selection-03 | | | | | | |
| GE-T0-Wc200-01 | 1 | Chloroplast | 1.8 | | | Variegated |
| GE-T0-Wc200-02 | 1 | Chloroplast | 0 | | | Variegated |
| GE-T0-Wc200-03 | 1 | Chloroplast | 0.2 | | | Variegated |
| GE-T0-Wc200-04 | 1 | Chloroplast | 0.2 | | | Variegated |
| GE-T0-Wc400-01 | 1 | Chloroplast | 0.9 | | | Variegated |
| GE-T0-Wc400-02 | 1 | Chloroplast | 0.6 | | | dead |
| GE-T0-Wc400-03 | 1 | Chloroplast | 0 | | | dead |
| GE-T0-Wm200-01 | 1 | Mitochondria | 0 | | | dead |
| GE-T0-Wm200-02 | 1 | Mitochondria | 0 | | | dead |
| GE-T0-Wm200-03 | 1 | Mitochondria | 0 | | | dead |
| GE-T0-Wm200-04 | 1 | Mitochondria | 0 | | | dead |
| GE-T0-Wm400-01 | 1 | Mitochondria | 0 | | | Variegated |
| GE-T0-Wm400-02 | 1 | Mitochondria | 0.3 | | | dead |
| GE-T0-Wm400-03 | 1 | Mitochondria | 0 | | | dead |
| GE-T0-Wm400-04 | 1 | Mitochondria | 0 | | | dead |
| control with | 2 | — | | 0 | 0 | Variegated |
| selection-01 | | | | | | |
| control with | 2 | — | | 0 | | dead |
| selection-02 | | | | | | |
| control without | 2 | — | | 0 | 0 | Variegated |
| selection-04 | | | | | | |
| GE-T0-Wc-01 | 2 | Chloroplast | | 1 | 0 | Variegated |
| GE-T0-Wc-02 | 2 | Chloroplast | | 1 | | dead |
| GE-T0-Wc-03 | 2 | Chloroplast | | 1 | | dead |
| GE-T0-Wc-04 | 2 | Chloroplast | | 805 | 6.3 | green and |
| | | | | | | healthy |
| GE-T0-Wc-05 | 2 | Chloroplast | | 1 | | dead |
| GE-T0-Wc-06 | 2 | Chloroplast | | 12 | 0 | Variegated |
| GE-T0-Wc-07 | 2 | Chloroplast | | 555 | 2.3 | green and |
| | | | | | | healthy |
| GE-T0-Wc-08 | 2 | Chloroplast | | 1137 | 0 | green and |
| | | | | | | healthy |
| GE-T0-Wc-09 | 2 | Chloroplast | | 5 | 0 | Variegated |
| GE-T0-Wc-10 | 2 | Chloroplast | | 1 | 0 | Variegated |
| GE-T0-Wc-11 | 2 | Chloroplast | | 1 | 0 | Variegated |
| GE-T0-Wc-12 | 2 | Chloroplast | | 2 | 0 | Variegated |
| GE-T0-Wm-01 | 2 | Mitochondria | | 261 | 0 | green and |
| | | | | | | healthy |
| GE-T0-Wm-02 | 2 | Mitochondria | | 12 | 0 | green and |
| | | | | | | healthy |
| GE-T0-Wm-03 | 2 | Mitochondria | | 8 | | dead |
| GE-T0-Wm-04 | 2 | Mitochondria | | 281 | 0.1 | green and |
| | | | | | | healthy |
| GE-T0-Wm-05 | 2 | Mitochondria | | 485 | 0.3 | green and |
| | | | | | | healthy |
| GE-T0-Wm-06 | 2 | Mitochondria | | 311 | 0.1 | green and |
| | | | | | | healthy |
| GE-T0-Wm-07 | 2 | Mitochondria | | 3 | 0 | Variegated |
| GE-T0-Wm-08 | 2 | Mitochondria | | 3 | 0 | Variegated |
| GE-T0-Wm-09 | 2 | Mitochondria | | 8 | 0 | Variegated |
| No viable plants | 3 | | | | | All plants |
| | | | | | | albino-few |
| | | | | | | plants |
| control with | 4 | — | | | | dead |
| selection-03 | | | | | | |
| control with | 4 | — | | | | dead |
| selection-04 | | | | | | |
| control with | 4 | — | | | | dead |
| selection-05 | | | | | | |
| control with | 4 | — | | | | dead |
| selection-06 | | | | | | |
| control with | 4 | — | | | | dead |
| selection-07 | | | | | | |
| control with | 4 | — | | | | dead |
| selection-08 | | | | | | |
| control with | 4 | — | | | | Variegated |
| selection-09 | | | | | | |
| control with | 4 | — | | | | Variegated |
| selection-10 | | | | | | |
| control with | 4 | — | | | | Variegated |
| selection-11 | | | | | | |
| control with | 4 | — | | | | Variegated |
| selection-12 | | | | | | |
| control with | 4 | — | | | | Variegated |
| selection-13 | | | | | | |
| control with | 4 | — | | | | Variegated |
| selection-14 | | | | | | |
| control with | 4 | — | | | | Variegated |
| selection-15 | | | | | | |
| control with | 4 | — | | | | Variegated |
| selection-16 | | | | | | |
| control with | 4 | — | | | | dead |
| selection-17 | | | | | | |
| control with | 4 | — | | | | dead |
| selection-18 | | | | | | |
| GE-T0-Wc-13 | 4 | Chloroplast | | | | dead |
| GE-T0-Wc-14 | 4 | Chloroplast | | | | Variegated |
| GE-T0-Wc-15 | 4 | Chloroplast | | | | Variegated |
| GE-T0-Wc-16 | 4 | Chloroplast | | | | Variegated |
| GE-T0-Wc-17 | 4 | Chloroplast | | | | Variegated |
| GE-T0-Wc-18 | 4 | Chloroplast | | | | green and |
| | | | | | | healthy |
| GE-T0-Wc-19 | 4 | Chloroplast | | | | Variegated |
| GE-T0-Wc-20 | 4 | Chloroplast | | | | dead |
| GE-T0-Wc-21 | 4 | Chloroplast | | | | dead |
| GE-T0-Wc-22 | 4 | Chloroplast | | | | dead |
| GE-T0-Wc-23 | 4 | Chloroplast | | | | dead |
| GE-T0-Wc-24 | 4 | Chloroplast | | | | Variegated |
| GE-T0-Wc-25 | 4 | Chloroplast | | | | dead |
| GE-T0-Wc-26 | 4 | Chloroplast | | | | dead |
| GE-T0-Wc-27 | 4 | Chloroplast | | | | dead |
| GE-T0-Wc-28 | 4 | Chloroplast | | | | dead |
| GE-T0-Wc-29 | 4 | Chloroplast | | | | dead |
| GE-T0-Wm-10 | 4 | Mitochondria | | | | green and |
| | | | | | | healthy |
| GE-T0-Wm-11 | 4 | Mitochondria | | | | dead |
| GE-T0-Wm-12 | 4 | Mitochondria | | | | dead |
| GE-T0-Wm-13 | 4 | Mitochondria | | | | green and |
| | | | | | | healthy |
| GE-T0-Wm-14 | 4 | Mitochondria | | | | dead |
| GE-T0-Wm-15 | 4 | Mitochondria | | | | Variegated |
| GE-T0-Wm-16 | 4 | Mitochondria | | | | dead |
| GE-T0-Wm-17 | 4 | Mitochondria | | | | Variegated |
| GE-T0-Wm-18 | 4 | Mitochondria | | | | dead |
| GE-T0-Wm-19 | 4 | Mitochondria | | | | dead |
| GE-T0-Wm-20 | 4 | Mitochondria | | | | dead |
| GE-T0-Wm-21 | 4 | Mitochondria | | | | dead |
| GE-T0-Wm-22 | 4 | Mitochondria | | | | dead |
| GE-T0-Wm-23 | 4 | Mitochondria | | | | dead |
| GE-T0-Wm-24 | 4 | Mitochondria | | | | dead |
| GE-T0-Wm-25 | 4 | Mitochondria | | | | dead |
| GE-T0-Wm-26 | 4 | Mitochondria | | | | dead |
| GE-T0-Wm-27 | 4 | Mitochondria | | | | dead |
| GE-T0-Wm-28 | 4 | Mitochondria | | | | green and |
| | | | | | | healthy |
| GE-T0-Wm-29 | 4 | Mitochondria | | | | dead |
| GE-T0-Wm-30 | 4 | Mitochondria | | | | Variegated |
| GE-T0-Wm-31 | 4 | Mitochondria | | | | dead |
| GE-T0-Wm-32 | 4 | Mitochondria | | | | green and |
| | | | | | | healthy |
| GE-T0-Wm-33 | 4 | Mitochondria | | | | green and |
| | | | | | | healthy |
| GE-T0-Wm-34 | 4 | Mitochondria | | | | green and |
| | | | | | | healthy |
| GE-T0-Wm-35 | 4 | Mitochondria | | | | dead |
| GE-T0-Wm-36 | 4 | Mitochondria | | | | dead |
| GE-T0-Wm-37 | 4 | Mitochondria | | | | dead |
| GE-T0-Wm-38 | 4 | Mitochondria | | | | dead |
| GE-T0-Wm-39 | 4 | Mitochondria | | | | dead |
| GE-T0-Wm-40 | 4 | Mitochondria | | | | dead |
| GE-T0-Wm-41 | 4 | Mitochondria | | | | dead |
| GE-T0-Wm-42 | 4 | Mitochondria | | | | dead |
| GE-T0-Wm-43 | 4 | Mitochondria | | | | green and |
| | | | | | | healthy |
| GE-T0-Wm-44 | 4 | Mitochondria | | | | green and |
| | | | | | | healthy |
| GE-T0-Wm-45 | 4 | Mitochondria | | | | dead |
| GE-T0-Wm-46 | 4 | Mitochondria | | | | green and |
| | | | | | | healthy |
| GE-T0-Wm-47 | 4 | Mitochondria | | | | dead |
| GE-T0-Wm-48 | 4 | Mitochondria | | | | dead |
| GE-T0-Wm-49 | 4 | Mitochondria | | | | dead |
| GE-T0-Wm-50 | 4 | Mitochondria | | | | dead |

The embodiments of the present invention described herein are intended to be illustrative and non-limiting. Various modifications which are readily apparent to the person of skill in the art are intended to be included. The scope of the claims should not be limited by the embodiments described herein but should be given the broadest interpretation consistent with the description as a whole.

1. A method for delivering a nucleic acid to a non-nuclear organelle in a cell, the method comprising exposing the cell to a composition comprising at least one nucleic acid and at least one organelle targeting nanocarrier; wherein the at least one nucleic acid translocates across a cell membrane of the cell and enters the non-nuclear organelle in the presence of the at least one organelle targeting nanocarrier.

2. The method according to claim 1 wherein the nucleic acid is DNA.

3. The method according to claim 1 wherein the cell is a plant cell.

4. The method according to claim 1 wherein the cell is an animal cell.

5. The method according to claim 1 wherein the non-nuclear organelle is a mitochondrion.

6. The method according to claim 5 wherein the organelle targeting nanocarrier is a polypeptide having a charge ratio of about 4 to about 7 and hydrophilicity of about 0 to about −0.5.

7. The method according to claim 6 wherein the organelle targeting nanocarrier is a polypeptide having a sequence selected from:

| | |
|---|---|
| MFSYLPRYPLRAASARALVRATRPSYRSALLRYQ; | (SEQ ID NO: 1) |
| MAAWMRSLFSPLKKLWIRMH; | (SEQ ID NO: 2) |
| MKLLWRLILSRKW; | (SEQ ID NO: 3) |
| MWWRRSRTNSLRYT; | (SEQ ID NO: 4) |
| and | |
| MLFRLRRSVRLRGLLA. | (SEQ ID NO: 5) |

8. The method according to claim 1 wherein the non-nuclear organelle is a chloroplast.

9. The method according to claim 8 wherein the organelle targeting nanocarrier is a polypeptide having a charge ratio of about 2 to about 4.2 and hydrophilicity of about 0 to about −0.2.

10. The method according to claim 9 wherein the organelle targeting nanocarrier is a polypeptide having a sequence selected from:

| | |
|---|---|
| MGGCVSTPKSCVGAKLR; | (SEQ ID NO: 6) |
| MQTLTASSSVSSIQRHRPHPAGRRSSSVTFS; | (SEQ ID NO: 7) |
| MKNPPSSFASGFGIR; | (SEQ ID NO: 8) |
| MAALIPAIASLPRAQVEKPHPMPVSTRPGLVS; | (SEQ ID NO: 9) |
| and | |
| MSSPPPLFTSCLPASSPSIRRDSTSGSVTSPLR. | (SEQ ID NO: 10) |

11. A method for producing a genetically modified plant cell, the method comprising: exposing a plant cell containing a non-nuclear organelle to a composition comprising at least one nucleic acid and at least one organelle targeting nanocarrier; wherein the at least one nucleic acid translocates across a cell membrane of the cell and enters the non-nuclear organelle in the presence of the at least one organelle targeting nanocarrier so as to transfect the non-nuclear organelle.

12. The method according to claim 11 wherein the plant cell is an embryogenic microspore.

13. A genetically modified plant cell produced by the method according to claim 11.

14. A method for producing a genetically modified plant, the method comprising:
producing a genetically modified plant cell by the method according to claim 11; and
generating a plant from the genetically modified plant cell.

15. A genetically modified plant generated by the method according to claim 14.

16. A method for producing a genetically modified animal cell, the method comprising:
exposing an animal cell containing at least one mitochondrion to a composition comprising at least one nucleic acid and at least one mitochondrion targeting nanocarrier; wherein the at least one nucleic acid translocates across a cell membrane of the cell and enters the at least one mitochondrion in the presence of the at least one mitochondrion targeting nanocarrier so as to transfect the at least one mitochondrion.

17. A mitochondrion targeting nanocarrier wherein the mitochondrion targeting nanocarrier is a polypeptide having a mitochondrial targeting peptide sequence, a charge ratio of about 4 to about 7 and a hydrophilicity of about 0 to about −0.5.

18. The mitochondrion targeting nanocarrier according to claim 17 wherein the polypeptide has a sequence selected from:

| | |
|---|---|
| MFSYLPRYPLRAASARALVRATRPSYRSALLRYQ; | (SEQ ID NO: 1) |
| MAAWMRSLFSPLKKLWIRMH; | (SEQ ID NO: 2) |
| MKLLWRLILSRKW; | (SEQ ID NO: 3) |
| MWWRRSRTNSLRYT; | (SEQ ID NO: 4) |
| and | |
| MLFRLRRSVRLRGLLA. | (SEQ ID NO: 5) |

19. A chloroplast targeting nanocarrier wherein the chloroplast targeting nanocarrier is a polypeptide having a chloroplast transit peptide sequence, a charge ratio of about 2 to about 4.2 and a hydrophilicity of about 0 to about −0.2.

20. The chloroplast targeting nanocarrier according to claim 19 wherein the polypeptide has a sequence selected from:

| | |
|---|---|
| MGGCVSTPKSCVGAKLR; | (SEQ ID NO: 6) |
| MQTLTASSSVSSIQRHRPHPAGRRSSSVTFS; | (SEQ ID NO: 7) |
| MKNPPSSFASGFGIR; | (SEQ ID NO: 8) |
| MAALIPAIASLPRAQVEKPHPMPVSTRPGLVS; | (SEQ ID NO: 9) |
| and | |
| MSSPPPLFTSCLPASSPSIRRDSTSGSVTSPLR. | (SEQ ID NO: 10) |


SEQUENCE LISTING (10 sequences)

SEQ ID NO: 1 - protein, 34 aa, Triticum aestivum
MFSYLPRYPLRAASARALVRATRPSYRSALLRYQ

SEQ ID NO: 2 - protein, 20 aa, Zea mays
MAAWMRSLFSPLKKLWIRMH

SEQ ID NO: 3 - protein, 13 aa, Arabidopsis thaliana
MKLLWRLILSRKW

SEQ ID NO: 4 - protein, 14 aa, Arabidopsis thaliana
MWWRRSRTNSLRYT

SEQ ID NO: 5 - protein, 16 aa, Arabidopsis thaliana
MLFRLRRSVRLRGLLA

SEQ ID NO: 6 - protein, 17 aa, Arabidopsis thaliana
MGGCVSTPKSCVGAKLR

SEQ ID NO: 7 - protein, 31 aa, Triticum aestivum
MQTLTASSSVSSIQRHRPHPAGRRSSSVTFS

SEQ ID NO: 8 - protein, 15 aa, Oryza sativa
MKNPPSSFASGFGIR

SEQ ID NO: 9 - protein, 32 aa, Triticum aestivum
MAALIPAIASLPRAQVEKPHPMPVSTRPGLVS

SEQ ID NO: 10 - protein, 33 aa, Arabidopsis thaliana
MSSPPPLFTSCLPASSPSIRRDSTSGSVTSPLR